(19) United States
(12) Patent Application Publication
(10) Pub. No.: US 2024/0254564 A1
(43) Pub. Date: Aug. 1, 2024

(54) METHODS FOR PREDICTING AND TREATING CHEMORESISTANCE IN SMALL CELL LUNG CANCER PATIENTS

(71) Applicants: MEMORIAL SLOAN-KETTERING CANCER CENTER, New York, NY (US); MEMORIAL HOSPITAL FOR CANCER AND ALLIED DISEASES, New York, NY (US); SLOAN-KETTERING INSTITUTE FOR CANCER RESEARCH, New York, NY (US)

(72) Inventors: Álvaro D. Quintanal Villalonga, New York, NY (US); Charles Michael Rudin, New York, NY (US); John Thomas Poirier, New York, NY (US)

(21) Appl. No.: 18/565,767

(22) PCT Filed: Jun. 1, 2022
(86) PCT No.: PCT/US22/31715

§ 371 Date: Nov. 30, 2023

Related U.S. Application Data
(60) Provisional application No. 63/196,820, filed on Jun. 4, 2021.

(51) Int. Cl. C12Q 1/6886; A61K 45/06

(52) U.S. Cl. CPC C12Q 1/6886; A61K 45/06; C12Q 2600/106; C12Q 2600/156

(57) ABSTRACT

The present disclosure relates to methods for detecting chemoresistant SCLC tumors in a patient and/or methods for determining whether a patient diagnosed with small cell lung cancer (SCLC) will benefit from treatment with chemotherapy. These methods are based on screening a SCLC patient for elevated XP01 expression. The present technology also provides methods for sensitizing SCLC patients to chemotherapy using an inhibitor of XP01.

### CROSS-REFERENCE TO RELATED APPLICATIONS

This application claims the benefit of and priority to U.S. Provisional Patent Application No. 63/196,820, filed Jun. 4, 2021, the entire contents of which are incorporated herein by reference.

### STATEMENT OF GOVERNMENT SUPPORT

This invention was made with government support under grant number CA197936 and CA213274 awarded by National Cancer Institute. The government has certain rights in the invention.

### TECHNICAL FIELD

The present technology relates to methods for detecting chemoresistant SCLC tumors in a patient and/or methods for determining whether a patient diagnosed with small cell lung cancer (SCLC) will benefit from treatment with chemotherapy. These methods are based on screening a SCLC patient for elevated XPO1 expression. The present technology also provides methods for sensitizing SCLC patients to chemotherapy using an inhibitor of XPO1. Kits for use in practicing the methods are also provided.

### BACKGROUND

The following description of the background of the present technology is provided simply as an aid in understanding the present technology and is not admitted to describe or constitute prior art to the present technology.

Small cell lung cancer (SCLC) is an extremely aggressive subtype of lung cancer comprising 13% of all lung cancer cases. With very limited treatment options that typically result in only short-term responses (Byers, L. A. & Rudin, C. M. Cancer 121, 664-672 (2015)), SCLC mortality is estimated as 250,000 deaths globally per year (Rudin, C. M. et al., Nat Rev Dis Primers 14, 1-20 (2021)). Despite decades of active research, the improvements in standard of care therapy have been sparse. SCLC treatment options include platinum-based doublet chemotherapy, to which SCLC tumors are initially sensitive. However, nearly all patients develop recurrent and chemoresistant disease, leading to median survival rates of around one year (Rudin, C. M. et al. J. Clin. Oncol. 33, 4106-4111 (2015)). The addition of immune checkpoint blockade to chemotherapy in the first line setting appears to benefit only a small subset of patients, with a modest increase in median overall survival of approximately 2 months (Horn, L. et al. N. Engl. J. Med. 379, 2220-2229 (2018)): chemotherapy remains the backbone of treatment. Major hurdles to improving SCLC treatment include development of rapid chemoresistance and ineffective second line therapies (Byers, L. A. & Rudin, C. M. Cancer 121, 664-672 (2015)). The identification of novel therapies showing higher efficacy is a major unmet clinical need.

### SUMMARY OF THE PRESENT TECHNOLOGY

In one aspect, the present disclosure provides a method for selecting a small cell lung cancer (SCLC) patient that has received or is receiving chemotherapy for treatment with an XPO1 inhibitor comprising (a) detecting the presence of at least one mutation that results in elevated expression or activity of exportin-1 in a biological sample obtained from the SCLC patient; and (b) administering an effective amount of an XPO1 inhibitor to the SCLC patient. In some embodiments, the XPO1 inhibitor is separately, sequentially or simultaneously administered with the chemotherapy. The at least one mutation may be an XPO1 missense mutation, or an increased copy number of XPO1 gene.

In another aspect, the present disclosure provides a method for sensitizing a SCLC patient to chemotherapy comprising administering to the SCLC patient an effective amount of an XPO1 inhibitor separately, sequentially or simultaneously with the chemotherapy, wherein the SCLC patient comprises at least one mutation that results in elevated expression or activity of exportin-1, optionally wherein the at least one mutation is detected in a biological sample obtained from the SCLC patient. The at least one mutation may be an XPO1 missense mutation, or an increased copy number of XPO1 gene.

Additionally or alternatively, in some embodiments of the methods disclosed herein, the at least one mutation is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

In yet another aspect, the present disclosure provides a method for sensitizing a SCLC patient to chemotherapy comprising administering to the SCLC patient an effective amount of an XPO1 inhibitor separately, sequentially or simultaneously with the chemotherapy, wherein mRNA and/or polypeptide expression and/or activity levels of exportin-1 in a biological sample obtained from the SCLC patient are elevated compared to that observed in a control sample obtained from a healthy subject or a predetermined threshold. In some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any and all embodiments of the methods disclosed herein, the biological sample obtained from the SCLC patient comprises biopsied tumor tissue, whole blood, plasma, or serum.

In one aspect, the present disclosure provides a method for treating SCLC in a patient in need thereof comprising administering to the patient an effective amount of an XPO1 inhibitor and an effective amount of chemotherapy. The XPO1 inhibitor and the chemotherapy may be administered separately, sequentially, or simultaneously. Additionally or alternatively, in some embodiments, exportin-1 mRNA and/or polypeptide expression and/or activity levels in the patient are elevated compared to a healthy subject or a predetermined threshold.

In any and all embodiments of the methods disclosed herein, the XPO1 inhibitor may be selected from the group consisting of leptomycin B (LMB), PKF050-638, CBS9106, a selective inhibitors of nuclear transport (SINE) compound, an inhibitory nucleic acid targeting XPO1, and an anti-exportin-1 neutralizing antibody. Examples of SINE compounds include, but are not limited to KPT-185, KPT-249, KPT-251, KPT-276, KPT-335, KPT-330 (Selinexor), SL-801 (felezonexor), or KPT-8602 (Eltanexor). In some embodiments, the inhibitory nucleic acid targeting XPO1 is a shRNA, a siRNA, a sgRNA, a ribozyme, or an anti-sense oligonucleotide.

In any of the preceding embodiments of the methods disclosed herein, the patient has not previously received chemotherapy, or is suffering from chemoresistant SCLC. In certain embodiments, the patient has a SCLC subtype selected from among ASCL1^{high}, NEUROD1^{high} POU2F3^{high} and YAP^{high}. Additionally or alternatively, in some embodiments, the patient exhibits stage I, stage II, stage III or stage IV SCLC.

In any and all embodiments of the methods disclosed herein, the chemotherapy may comprise one or more chemotherapeutic agents. Examples of chemotherapeutic agents useful in the methods of the present technology include, but are not limited to antimetabolites, DNA alkylating agents, platinum agents, taxanes, topoisomerase inhibitors, endoplasmic reticulum stress inducing agents, anti-tumor antibiotics, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and combinations thereof.

In any of the preceding embodiments of the methods disclosed herein, the XPO1 inhibitor is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically. Additionally or alternatively, in some embodiments of the methods disclosed herein, the one or more chemotherapeutic agents are administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically. In any and all embodiments of the methods disclosed herein, the patient is human.

In one aspect, the present disclosure provides a method for detecting chemoresistant SCLC tumors in a patient in need thereof comprising detecting the presence of at least one mutation that results in elevated expression or activity of exportin-1 in a biological sample obtained from the patient, and/or detecting mRNA and/or polypeptide expression and/or activity levels of exportin-1 in a biological sample obtained from the patient that are elevated compared to a control sample obtained from a healthy subject or a predetermined threshold. The at least one mutation may be detected using any nucleic acid detection assay known in the art such as next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In certain embodiments, the at least one mutation may be an XPO1 missense mutation (e.g., E571, R749, and D624), or an increased copy number of XPO1 gene. In some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry. Additionally or alternatively, in some embodiments, the biological sample comprises polypeptides, genomic DNA, cDNA, RNA, and/or mRNA.

Also disclosed herein are kits comprising an XPO1 inhibitor, one or more chemotherapeutic agents, and instructions for treating SCLC.

### BRIEF DESCRIPTION OF THE DRAWINGS

FIGS. 1A-1G show that CRISPR screening identified XPO1 as a target for cisplatin sensitization. FIG. 1A shows the layout of the CRISPR screen. FIG. 1B shows Cisplatin G150 for the SCLC cell lines used in the Examples described herein. ASCL1^{high} SCLC: H69, Lx101 and Lx110; NEUROD1^{high} SCLC: Lx33 and H82; POU2F3^{high} SCLC: H526; and YAP^{high} SCLC: DMS114. FIG. 1C shows a plot demonstrating the genes for which sgRNAs were depleted on the highest numbers of SCLC cell lines in the cisplatin-treated condition versus the control condition. ASCL1^{high} SCLC: H69, Lx101 and Lx110; NEUROD1^{high} SCLC: Lx33 and H82; POU2F3^{high} SCLC: H526; and YAP^{high} SCLC: DMS114. FIG. 1D shows a heat-map demonstrating depletion levels of sgRNAs against genes shown in FIG. 1C in each cell line screened. ASCL1^{high} SCLC: H69, Lx101 and Lx110; NEUROD1^{high} SCLC: Lx33 and H82; POU2F3^{high} SCLC: H526; and YAP^{high} SCLC: DMS114. FIG. 1E shows a Western blot demonstrating XPO1 expression in Cas9-expressing H69 and H82 SCLC cell lines transduced with vectors expressing two different sgRNAs against XPO1. FIG. 1F shows clonal competition assays in vitro demonstrating variation in frequency of the cell populations under study, for control and XPO1-KO cell lines+/−cisplatin (GI20). Population percentages at the different time points were normalized to those of day 0. FIG. 1G shows an in vivo clonal competition assay in the inducible Cas9 PDX model Lx33 treated with cisplatin. Population percentages at endpoint were normalized to those of the non doxycycline-treated (non Cas9-expressing) condition. Two tailed Student's t-test was used to assess statistical significance of the differential expression between groups. p-values legend: * p<0.05, ** p<0.01, *** p<0.001.

FIGS. 2A-2E show that Exportin-1 inhibition exerts synergistic effects in combination with cisplatin in vitro. FIG. 2A shows synergy plots demonstrating the occurrence of synergy, addition or antagonism of the different combinations of Exportin-1 inhibitors (Selinexor and KPT-185) and cisplatin concentrations, calculated with the Highest single agent (HSA) method using the SynergyFinder web application (2.0). HSA synergy scores for H69 Cisplatin+Selinexor and H69 Cisplatin+KPT-185 are 6.55 and 8.52, respectively. HSA synergy scores for H82 Cisplatin+Selinexor and H82 Cisplatin+KPT-185 are 12.29 and 10.68, respectively. FIG. 2B shows a Selinexor specificity assay demonstrating viability (normalized to untreated condition) of endogenously XPO1-expressing (sgSAFE) and XPO1-KO (sgXPO1) cells. The sequence of the sgXPO1 is AACCTGAACGAAATGCCTG (SEQ ID NO: 16). FIG. 2C shows an outline of the rescue assay experiment. FIG. 2D shows a Western blot demonstrating XPO1 protein expression in cell lines with or without XPO1 KO and with or without XPO1-GFP re-expression. FIG. 2E shows rescue assay results demonstrating cell viability (normalized to untreated condition) of cisplatin-treated (G120) cell lines described in FIG. 2D. Two tailed Student's t-test was used to assess statistical significance of the differential expression between groups. p-values legend: * p<0.05, ** p<0.01, *** p<0.001.

FIGS. 3A-3C show that Exportin-1 inhibition increases apoptosis and DNA damage in combination with cisplatin. FIGS. 3A-3B show bar plots demonstrating the percentages of healthy and apoptotic cells, as determined by annexin V and PI assay by flow cytometry, in cisplatin-treated H82 cells with or without pharmacological (FIG. 3A) or genetic (FIG. 3B) Exportin-1 inhibition. FIG. 3C shows a Western blot demonstrating activation or expression levels of proteins associated to DNA damage sensing and repair, in cisplatin-treated H82 cells with or without pharmacological or genetic Exportin-1 inhibition. Two tailed Student's t-test was used to assess statistical significance of the differential expression between groups. p-values legend: * p<0.05, ** p<0.01, *** p<0.001.

FIGS. 4A-4D show that Exportin-1 is highly expressed in SCLC and its inhibition in combination with cisplatin is highly effective in chemonaive SCLC PDXs. FIGS. 4A-4B show XPO1 mRNA expression in cell lines derived from different tumor types (FIG. 4A) and in SCLC cell lines divided by SCLC subtype (FIG. 4B). The data was obtained from CCLE through UCSC Xenabrowser portal (xenabrowser.net) on December 2020. FIG. 4C shows Exportin-1 protein expression assessed by IHC in NSCLC (N=58) and SCLC (N=32) clinical samples. Protein expression is shown as IHC score. FIG. 4D shows graphs demonstrating tumor growth of chemonaive SCLC PDXs treated with cisplatin, etoposide, Selinexor and their multiple combinations. Two tailed Student's t-test was used to assess statistical significance of the differential expression between groups. p-values legend: * p<0.05, ** p<0.01, *** p<0.001.

FIGS. 5A-5B show that Exportin-1 inhibition exerts synergistic effects in combination with irinotecan. FIG. 5A shows synergy plots demonstrating the occurrence of synergy (red), addition (white) or antagonism (green) of the different combinations of exportin-1 inhibitors (Selinexor and KPT-185) and irinotecan concentrations, calculated with the HSA method using the SynergyFinder web application (2.0). FIG. 5B shows graphs demonstrating tumor growth of SCLC PDXs derived from chemorelapsed tumors treated with irinotecan, Selinexor, or their combination. Two tailed Student's t-test was used to assess statistical significance of the differential expression between groups. p-values legend: * p<0.05, ** p<0.01, *** p<0.001.

FIGS. 6A-6B show bar plots demonstrating the percentages of healthy and apoptotic cells, detailed as healthy, early apoptotic, late apoptotic or necrotic cells for cisplatin-treated H82 cells with or without pharmacological (FIG. 6A) or genetic (FIG. 6B) Exportin-1 inhibition. FIG. 6C shows Western blot quantifications of pATR/ATR, pATM/ATM and pChk2/Chk2 in cisplatin-treated H82 cells with or without pharmacological or genetic Exportin-1 inhibition. Two tailed Student's t-test was used to assess statistical significance of the differential expression between groups. p-values legend: * p<0.05, ** p<0.01, *** p<0.001.

FIG. 7A shows body weight of mice over time treated with cisplatin, etoposide, Selinexor and their multiple combinations. FIG. 7B shows graphs demonstrating tumor growth of an additional chemonaive SCLC PDX treated with cisplatin, etoposide, Selinexor and their multiple combinations. Two tailed Student's t-test was used to assess statistical significance of the differential expression between groups. p-values legend: * p<0.05, ** p<0.01, *** p<0.001.

FIG. 8A shows a graph demonstrating tumor growth over time of a chemoresistant SCLC PDX generated in vivo by continuous treatment with cisplatin and etoposide, treated with irinotecan, Selinexor, or their combination. FIG. 8B shows body weight of mice treated with irinotecan, Selinexor or their combination over time. FIG. 8C shows graphs demonstrating tumor growth of an additional SCLC PDX derived from a chemorelapsed tumor treated with irinotecan, Selinexor or their combination over time. Two tailed Student's t-test was used to assess statistical significance of the differential expression between groups. p-values legend: * p<0.05, ** p<0.01, *** p<0.001.

FIGS. 9A-9G show impairment of chemotherapy-induced AKT/mTOR overactivation may contribute to selinexor efficacy in combination with chemotherapy. FIG. 9A shows common pathways with downregulated differentially expressed genes in three SCLC models treated with selinexor. FIG. 9B shows pathways related to cholesterol synthesis and AKT/mTOR signaling, with genes differentially downregulated in the cisplatin+selinexor-treated versus cisplatin-treated SCLC models. FIG. 9C shows western blots assessing AKT/mTOR pathway in H69 and H82 cell lines treated with cisplatin (0.1 mmol/L), selinexor (0.05 mmol/L), or their combination for 7 days. FIG. 9D shows synergy plots for different combinations of concentrations of cisplatin and the AKT/mTOR inhibitor samotolisib. FIG. 9E shows pathways related to cholesterol synthesis and AKT/mTOR signaling, with genes differentially downregulated in the irinotecan+selinexor-treated versus irinotecan-treated Lx1322 SCLC PDX. FIG. 9F shows western blots showing activation of AKT/mTOR pathway in H69 and H82 cell lines treated with irinotecan (0.05 mmol/L), selinexor (0.05 mmol/L), or their combination for 7 days. FIG. 9G shows synergy plots for different combinations of concentrations of irinotecan and the AKT/mTOR inhibitor samotolisib. Synergy plots, showing the occurrence of synergy, addition, or antagonism (green), were calculated with the HSA method using the SynergyFinder web application (2.0).

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

Improvements in standard of care therapy for SCLC have been sparse. Nearly all SCLC patients develop recurrent and chemoresistant disease, leading to median survival rates of around one year. CRISPR-Cas9 screening has been previously applied to POU2F3^{high} non-neuroendocrine cell lines (Huang, Y. H. et al. Genes Dev. 32, 915-928 (2018)), which represent a minority of SCLC cases, with limited success in preclinical models of the most prevalent SCLC subtypes, ASCL1^{high} and NEUROD1^{high}.

The present disclosure successfully applies CRISPR-Cas9 screening in SCLC cell lines and short-term cultured patient-derived xenograft (PDX)-derived cell lines representing all SCLC subtypes, and identifies the nuclear export protein Exportin-1 (encoded by the XPO1 gene), as a novel and highly effective combination pharmacological target in chemonaive and therapy-relapsed SCLC.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3SR), including multiplex versions or combinations thereof. Copies of a particular nucleic acid sequence generated in vitro in an amplification reaction are called “amplicons” or “amplification products.”

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “subject”, “patient”, or “individual” can be an individual organism, a vertebrate, a mammal, or a human. In some embodiments, the subject, patient or individual is a human.

As used herein, the term “therapeutic agent” is intended to mean a compound that, when present in an effective amount, produces a desired therapeutic effect on a subject in need thereof.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Methods for Detecting Polynucleotides Associated with Chemoresistant SCLC Tumors

Polynucleotides associated with chemoresistant SCLC tumors may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

Nucleic Acid Amplification and/or Detection

Polynucleotides associated with responsiveness to intraoperative opioid analgesics can be detected by the use of nucleic acid amplification techniques that are well known in the art. The starting material may be genomic DNA, cDNA, RNA or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif, 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding an Exportin-1 polypeptide. Exemplary nucleic acid sequences of the human orthologs of these genes are provided below:

>XM_011533097.1 PREDICTED: Homo sapiens exportin 1 (XPO1),
transcript variant X4, mRNA
(SEQ ID NO: 1)
CACTCTGCGGGCAAATCGCTGCTGAAAAGAGTAATCTATGCCAGCAATTATGACAATGTTAGCAGACCAT
GCAGCTCGTCAGCTGCTTGATTTCAGCCAAAAACTGGATATCAACTTATTAGATAATGTGGTGAATTGCT
TATACCATGGAGAAGGAGCCCAGCAAAGAATGGCTCAAGAAGTACTGACACATTTAAAGGAGCATCCTGA
TGCTTGGACAAGAGTCGACACAATTTTGGAATTTTCTCAGAATATGAATACGAAATACTATGGACTACAA
ATTTTGGAAAATGTGATAAAAACAAGGTGGAAGATTCTTCCAAGGAACCAGTGCGAAGGAATAAAAAAAT
ACGTTGTTGGCCTCATTATCAAGACGTCATCTGACCCAACTTGTGTAGAGAAAGAAAAGGTGTATATCGG
AAAATTAAATATGATCCTTGTTCAGATACTGAAACAAGAATGGCCCAAACATTGGCCAACTTTTATCAGT
GATATTGTTGGAGCAAGTAGGACCAGCGAAAGTCTCTGTCAAAATAATATGGTGATTCTTAAACTCTTGA
GTGAAGAAGTATTTGATTTCTCTAGTGGACAGATAACCCAAGTCAAATCTAAGCATTTAAAAGACAGCAT
GTGCAATGAATTCTCACAGATATTTCAACTGTGTCAGTTTGTAATGGAAAATTCTCAAAATGCTCCACTT
GTACATGCAACCTTGGAAACATTGCTCAGATTTCTGAACTGGATTCCCCTGGGATATATTTTTGAGACCA
AATTAATCAGCACATTGATTTATAAGTTCCTGAATGTTCCAATGTTTCGAAATGTCTCTCTGAAGTGCCT
CACTGAGATTGCTGGTGTGAGTGTAAGCCAATATGAAGAACAATTTGTAACACTATTTACTCTGACAATG
ATGCAACTAAAGCAGATGCTTCCTTTAAATACCAATATTCGACTTGCGTACTCAAATGGAAAAGATGATG
AACAGAACTTCATTCAAAATCTCAGTTTGTTTCTCTGCACCTTTCTTAAGGAACATGATCAACTTATAGA
AAAAAGATTAAATCTCAGGGAAACTCTTATGGAGGCCCTTCATTATATGTTGTTGGTATCTGAAGTAGAA
GAAACTGAAATCTTTAAAATTTGTCTTGAATACTGGAATCATTTGGCTGCTGAACTCTATAGAGAGAGTC
CATTCTCTACATCTGCCTCTCCGTTGCTTTCTGGAAGTCAACATTTTGATGTTCCTCCCAGGAGACAGCT
ATATTTGCCCATGTTATTCAAGGTCCGTTTATTAATGGTTAGTCGAATGGCTAAACCAGAGGAAGTATTG
GTTGTAGAGAATGATCAAGGAGAAGTTGTGAGAGAATTCATGAAGGATACAGATTCCATAAATTTGTATA
AGAATATGAGGGAAACATTGGTTTATCTTACTCATCTGGATTATGTAGATACAGAAAGAATAATGACAGA
GAAGCTTCACAATCAAGTGAATGGTACAGAGTGGTCATGGAAAAATTTGAATACATTGTGTTGGGCAATA
GGCTCCATTAGTGGAGCAATGCATGAAGAGGACGAAAAACGATTTCTTGTTACTGTTATAAAGGATCTAT
TAGGATTATGTGAACAGAAAAGAGGCAAAGATAATAAAGCTATTATTGCATCAAATATCATGTACATAGT
AGGTCAATACCCACGTTTTTTGAGAGCTCACTGGAAATTTCTGAAGACTGTAGTTAACAAGCTGTTCGAA
TTCATGCATGAGACCCATGATGGAGTCCAGGATATGGCTTGTGATACTTTCATTAAAATAGCCCAAAAAT
GCCGCAGGCATTTCGTTCAGGTTCAGGTTGGAGAAGTGATGCCATTTATTGATGAAATTTTGAACAACAT
TAACACTATTATTTGTGATCTTCAGCCTCAACAGGTTCATACGTTTTATGAAGCTGTGGGGTACATGATT
GGTGCACAAACAGATCAAACAGTACAAGAACACTTGATAGAAAAGTACATGTTACTCCCTAATCAAGTGT
GGGATAGTATAATCCAGCAGGCAACCAAAAATGTGGATATACTGAAAGATCCTGAAACAGTCAAGCAGCT
TGGTAGCATTTTGAAAACAAATGTGAGAGCCTGCAAAGCTGTTGGACACCCCTTTGTAATTCAGCTTGGA
AGAATTTATTTAGATATGCTTAATGTATACAAGTGCCTCAGTGAAAATATTTCTGCAGCTATCCAAGCTA
ATGGTGAAATGGTTACAAAGCAACCATTGATTAGAAGTATGCGAACTGTAAAAAGGGAAACTTTAAAGTT
AATATCTGGTTGGGTGAGCCGATCCAATGATCCACAGATGGTCGCTGAAAATTTTGTTCCCCCTCTGTTG
GATGCAGTTCTCATTGATTATCAGAGAAATGTCCCAGCTGCTAGAGAACCAGAAGTGCTTAGTACTATGG
CCATAATTGTCAACAAGTTAGGGGGACATATAACAGCTGAAATACCTCAAATATTTGATGCTGTTTTTGA
ATGCACATTGAATATGATAAATAAGGACTTTGAAGAATATCCTGAACATAGAACGAACTTTTTCTTACTA
CTTCAGGCTGTCAATTCTCATTGTTTCCCAGCATTCCTTGCTATTCCACCTACACAGTTTAAACTTGTTT
TGGATTCCATCATTTGGGCTTTCAAACATACTATGAGGAATGTCGCAGATACGGGCTTACAGATACTTTT
TACACTCTTACAAAATGTTGCACAAGAAGAAGCTGCAGCTCAGAGTTTTTATCAAACTTATTTTTGTGAT
ATTCTCCAGCATATCTTTTCTGTTGTGACAGACACTTCACATACTGCTGGTTTAACAATGCATGCATCAA
TTCTTGCATATATGTTTAATTTGGTTGAAGAAGGAAAAATAAGTACATCATTAAATCCTGGAAATCCAGT
TAACAACCAAATCTTTCTTCAGGAATATGTGGCTAATCTCCTTAAGTCGGCCTTCCCTCACCTACAAGAT
GCTCAAGTAAAGCTCTTTGTGACAGGGCTTTTCAGCTTAAATCAAGATATTCCTGCTTTCAAGGAACATT
TAAGAGATTTCCTAGTTCAAATAAAGGAATTTGCAGGTGAAGACACTTCTGATTTGTTTTTGGAAGAGAG
AGAAATAGCCCTACGGCAGGCTGATGAAGAGAAACATAAACGTCAAATGTCTGTCCCTGGCATCTTTAAT
CCACATGAGATTCCAGAAGAAATGTGTGATTAAAATCCAAATTCATGCTGTTTTTTTTCTCTGCAACTCG
TTAGCAGAGGAAAACAGCATGTGGGTATTTGTCGACCAAAATGATGCCAATTTGTAAATTAAAATGTCAC
CTAGTGGCCCTTTTTCTTATGTGTTTTTTTGTATAAGAAATTTTCTGTGAAATATCCTTCCATTGTTTAA
GCTTTTGTTTTGGTCATCTTTATTTAGTTTGCATGAAGTTGAAAATTAAGGCATTTTTAAAAATTTTACT
TCATGCCCATTTTTGTGGCTGGGCTGGGGGGAGGAGGCAAATTCGATTTGAACATATACTTGTAATTCTA
ATGCAAAATTATACAATTTTTCCTGTAAACAATACCAATTTTTAATTAGGGAGCATTTTCCTTCTAGTCT
ATTTCAGCCTAGAAGAAAAGATAATGAGTAAAACAAATTGCGTTGTTTAAAGGATTATAGTGCTGCATTG
TCTGAAGTTAGCACCTCTTGGACTGAATCGTTTGTCTAGACTACATGTATTACAAAGTCTCTTTGGCAAG
ATTGCAGCAAGATCATGTGCATATCATCCCATTGTAAAGCGACTTCAAAAATATGGGAACACAGTTAGTT
ATTTTTACACAGTTCTTTTTGTTTTTGTGTGTGTGTGCTGTCGCTTGTCGACAACAGCTTTTTGTTTTCC
TCAATGAGGAGTGTTGCTCATTTGTGAGCCTTCATTAACTCGAAGTGAAATGGTTAAAAATATTTATCCT
GTTAGAATAGGCTGCATCTTTTTAACAACTCATTAAAAAACAAAACAACTCTGGCTTTTGAGATGACTTA
TACTAATTTACATTGTTTACCAAGCTGTAGTGCTTTAAGAACACTACTTAAAAAGCAAAATAAACTTGGT
TTACATTTA
>XM_005264544.2 PREDICTED: Homo sapiens exportin 1 (XPO1),
transcript variant X5, mRNA
(SEQ ID NO: 2)
AGCGCCGGAGCCGCGTGAGAGAGGGAGCCGTGTTTTGGTAGGGGGGAGTCGGACTGCAACTGGCAGCAGA
GCGTCTCCCCGGCCGTGTGGACTCTACACCCCCTACTCCTGCCGCTTCTGCTGCTGCCTGTGGCTGGAGG
GTCCCCCTGGGGCTGAATCTTTGGGACTTGACCCCGTTCCCTCCCCCTTCCCTCACTCCCCAGCCGGGCG
GGAGCATTTATTCCCCAGATTAATTCCCCTTTTGGGGGGGGGGGGGTGTGTGTGTGTGTGTGTGTGTGTG
TGTGTGTTGGGGGAAGCGTCCCTGAAATAGTAAATATTATTGAGCTCTTTTTGCCCTTTTCCTGTCCGTT
TTTTTAATTTCCTTTTTTGAGGTGGGAAAACTGAAACCCACCTTGATTCGTCCCCTCTCCCCCCTCCCCA
CCTTCCCTCGCCCTAATCCCCCAACGAGGAAGGAAGGAGCAGTTGGTTCAATCTCTGGTAATCTATGCCA
GCAATTATGACAATGTTAGCAGACCATGCAGCTCGTCAGCTGCTTGATTTCAGCCAAAAACTGGATATCA
ACTTATTAGATAATGTGGTGAATTGCTTATACCATGGAGAAGGAGCCCAGCAAAGAATGGCTCAAGAAGT
ACTGACACATTTAAAGGAGCATCCTGATGCTTGGACAAGAGTCGACACAATTTTGGAATTTTCTCAGAAT
ATGAATACGAAATACTATGGACTACAAATTTTGGAAAATGTGATAAAAACAAGGTGGAAGATTCTTCCAA
GGAACCAGTGCGAAGGAATAAAAAAATACGTTGTTGGCCTCATTATCAAGACGTCATCTGACCCAACTTG
TGTAGAGATACTGAAACAAGAATGGCCCAAACATTGGCCAACTTTTATCAGTGATATTGTTGGAGCAAGT
AGGACCAGCGAAAGTCTCTGTCAAAATAATATGGTGATTCTTAAACTCTTGAGTGAAGAAGTATTTGATT
TCTCTAGTGGACAGATAACCCAAGTCAAATCTAAGCATTTAAAAGACAGCATGTGCAATGAATTCTCACA
GATATTTCAACTGTGTCAGTTTGTAATGGAAAATTCTCAAAATGCTCCACTTGTACATGCAACCTTGGAA
ACATTGCTCAGATTTCTGAACTGGATTCCCCTGGGATATATTTTTGAGACCAAATTAATCAGCACATTGA
TTTATAAGTTCCTGAATGTTCCAATGTTTCGAAATGTCTCTCTGAAGTGCCTCACTGAGATTGCTGGTGT
GAGTGTAAGCCAATATGAAGAACAATTTGTAACACTATTTACTCTGACAATGATGCAACTAAAGCAGATG
CTTCCTTTAAATACCAATATTCGACTTGCGTACTCAAATGGAAAAGATGATGAACAGAACTTCATTCAAA
ATCTCAGTTTGTTTCTCTGCACCTTTCTTAAGGAACATGATCAACTTATAGAAAAAAGATTAAATCTCAG
GGAAACTCTTATGGAGGCCCTTCATTATATGTTGTTGGTATCTGAAGTAGAAGAAACTGAAATCTTTAAA
ATTTGTCTTGAATACTGGAATCATTTGGCTGCTGAACTCTATAGAGAGAGTCCATTCTCTACATCTGCCT
CTCCGTTGCTTTCTGGAAGTCAACATTTTGATGTTCCTCCCAGGAGACAGCTATATTTGCCCATGTTATT
CAAGGTCCGTTTATTAATGGTTAGTCGAATGGCTAAACCAGAGGAAGTATTGGTTGTAGAGAATGATCAA
GGAGAAGTTGTGAGAGAATTCATGAAGGATACAGATTCCATAAATTTGTATAAGAATATGAGGGAAACAT
TGGTTTATCTTACTCATCTGGATTATGTAGATACAGAAAGAATAATGACAGAGAAGCTTCACAATCAAGT
GAATGGTACAGAGTGGTCATGGAAAAATTTGAATACATTGTGTTGGGCAATAGGCTCCATTAGTGGAGCA
ATGCATGAAGAGGACGAAAAACGATTTCTTGTTACTGTTATAAAGGATCTATTAGGATTATGTGAACAGA
AAAGAGGCAAAGATAATAAAGCTATTATTGCATCAAATATCATGTACATAGTAGGTCAATACCCACGTTT
TTTGAGAGCTCACTGGAAATTTCTGAAGACTGTAGTTAACAAGCTGTTCGAATTCATGCATGAGACCCAT
GATGGAGTCCAGGATATGGCTTGTGATACTTTCATTAAAATAGCCCAAAAATGCCGCAGGCATTTCGTTC
AGGTTCAGGTTGGAGAAGTGATGCCATTTATTGATGAAATTTTGAACAACATTAACACTATTATTTGTGA
TCTTCAGCCTCAACAGGTTCATACGTTTTATGAAGCTGTGGGGTACATGATTGGTGCACAAACAGATCAA
ACAGTACAAGAACACTTGATAGAAAAGTACATGTTACTCCCTAATCAAGTGTGGGATAGTATAATCCAGC
AGGCAACCAAAAATGTGGATATACTGAAAGATCCTGAAACAGTCAAGCAGCTTGGTAGCATTTTGAAAAC
AAATGTGAGAGCCTGCAAAGCTGTTGGACACCCCTTTGTAATTCAGCTTGGAAGAATTTATTTAGATATG
CTTAATGTATACAAGTGCCTCAGTGAAAATATTTCTGCAGCTATCCAAGCTAATGGTGAAATGGTTACAA
AGCAACCATTGATTAGAAGTATGCGAACTGTAAAAAGGGAAACTTTAAAGTTAATATCTGGTTGGGTGAG
CCGATCCAATGATCCACAGATGGTCGCTGAAAATTTTGTTCCCCCTCTGTTGGATGCAGTTCTCATTGAT
TATCAGAGAAATGTCCCAGCTGCTAGAGAACCAGAAGTGCTTAGTACTATGGCCATAATTGTCAACAAGT
TAGGGGGACATATAACAGCTGAAATACCTCAAATATTTGATGCTGTTTTTGAATGCACATTGAATATGAT
AAATAAGGACTTTGAAGAATATCCTGAACATAGAACGAACTTTTTCTTACTACTTCAGGCTGTCAATTCT
CATTGTTTCCCAGCATTCCTTGCTATTCCACCTACACAGTTTAAACTTGTTTTGGATTCCATCATTTGGG
CTTTCAAACATACTATGAGGAATGTCGCAGATACGGGCTTACAGATACTTTTTACACTCTTACAAAATGT
TGCACAAGAAGAAGCTGCAGCTCAGAGTTTTTATCAAACTTATTTTTGTGATATTCTCCAGCATATCTTT
TCTGTTGTGACAGACACTTCACATACTGCTGGTTTAACAATGCATGCATCAATTCTTGCATATATGTTTA
ATTTGGTTGAAGAAGGAAAAATAAGTACATCATTAAATCCTGGAAATCCAGTTAACAACCAAATCTTTCT
TCAGGAATATGTGGCTAATCTCCTTAAGTCGGCCTTCCCTCACCTACAAGATGCTCAAGTAAAGCTCTTT
GTGACAGGGCTTTTCAGCTTAAATCAAGATATTCCTGCTTTCAAGGAACATTTAAGAGATTTCCTAGTTC
AAATAAAGGAATTTGCAGGTGAAGACACTTCTGATTTGTTTTTGGAAGAGAGAGAAATAGCCCTACGGCA
GGCTGATGAAGAGAAACATAAACGTCAAATGTCTGTCCCTGGCATCTTTAATCCACATGAGATTCCAGAA
GAAATGTGTGATTAAAATCCAAATTCATGCTGTTTTTTTTCTCTGCAACTCGTTAGCAGAGGAAAACAGC
ATGTGGGTATTTGTCGACCAAAATGATGCCAATTTGTAAATTAAAATGTCACCTAGTGGCCCTTTTTCTT
ATGTGTTTTTTTGTATAAGAAATTTTCTGTGAAATATCCTTCCATTGTTTAAGCTTTTGTTTTGGTCATC
TTTATTTAGTTTGCATGAAGTTGAAAATTAAGGCATTTTTAAAAATTTTACTTCATGCCCATTTTTGTGG
CTGGGCTGGGGGGAGGAGGCAAATTCGATTTGAACATATACTTGTAATTCTAATGCAAAATTATACAATT
TTTCCTGTAAACAATACCAATTTTTAATTAGGGAGCATTTTCCTTCTAGTCTATTTCAGCCTAGAAGAAA
AGATAATGAGTAAAACAAATTGCGTTGTTTAAAGGATTATAGTGCTGCATTGTCTGAAGTTAGCACCTCT
TGGACTGAATCGTTTGTCTAGACTACATGTATTACAAAGTCTCTTTGGCAAGATTGCAGCAAGATCATGT
GCATATCATCCCATTGTAAAGCGACTTCAAAAATATGGGAACACAGTTAGTTATTTTTACACAGTTCTTT
TTGTTTTTGTGTGTGTGTGCTGTCGCTTGTCGACAACAGCTTTTTGTTTTCCTCAATGAGGAGTGTTGCT
CATTTGTGAGCCTTCATTAACTCGAAGTGAAATGGTTAAAAATATTTATCCTGTTAGAATAGGCTGCATC
TTTTTAACAACTCATTAAAAAACAAAACAACTCTGGCTTTTGAGATGACTTATACTAATTTACATTGTTT
ACCAAGCTGTAGTGCTTTAAGAACACTACTTAAAAAGCAAAATAAACTTGGTTTACATTTA
>XM_011533098.2 PREDICTED: Homo sapiens exportin 1
(XPO1), transcript variant X6, mRNA
(SEQ ID NO: 3)
AGCGCCGGAGCCGCGTGAGAGAGGGAGCCGTGTTTTGGTAGGGGGGAGTCGGACTGCAACTGGCAGCAGA
GCGTCTCCCCGGCCGTGTGGACTCTACACCCCCTACTCCTGCCGCTTCTGCTGCTGCCTGTGGCTGGAGG
GTCCCCCTGGGGCTGAATCTTTGGGACTTGACCCCGTTCCCTCCCCCTTCCCTCACTCCCCAGCCGGGCG
GGAGCATTTATTCCCCAGATTAATTCCCCTTTTGGGGGGGGGGGGGTGTGTGTGTGTGTGTGTGTGTGTG
TGTGTGTTGGGGGAAGCGTCCCTGAAATAGTAAATATTATTGAGCTCTTTTTGCCCTTTTCCTGTCCGTT
TTTTTAATTTCCTTTTTTGAGGTGGGAAAACTGAAACCCACCTTGATTCGTCCCCTCTCCCCCCTCCCCA
CCTTCCCTCGCCCTAATCCCCCAACGAGGAAGGAAGGAGCAGTTGGTTCAATCTCTGGTAATCTATGCCA
GCAATTATGACAATGTTAGCAGACCATGCAGCTCGTCAGCTGCTTGATTTCAGCCAAAAACTGGATATCA
ACTTATTAGATAATGTGGTGAATTGCTTATACCATGGAGAAGGAGCCCAGCAAAGAATGGCTCAAGAAGT
ACTGACACATTTAAAGGAGCATCCTGATGCTTGGACAAGAGTCGACACAATTTTGGAATTTTCTCAGAAT
ATGAATACGAAATACTATGGACTACAAATTTTGGAAAATGTGATAAAAACAAGGTGGAAGATTCTTCCAA
GGAACCAGTGCGAAGGAATAAAAAAATACGTTGTTGGCCTCATTATCAAGACGTCATCTGACCCAACTTG
TGTAGAGAAAGAAAAGGTGTATATCGGAAAATTAAATATGATCCTTGTTCAGATACTGAAACAAGAATGG
CCCAAACATTGGCCAACTTTTATCAGTGATATTGTTGGAGCAAGTAGGACCAGCGAAAGTCTCTGTCAAA
ATAATATGGTGATTCTTAAACTCTTGAGTGAAGAAGTATTTGATTTCTCTAGTGGACAGATAACCCAAGT
CAAATCTAAGCATTTAAAAGACAGCATGTGCAATGAATTCTCACAGATATTTCAACTGTGTCAGTTTGTA
ATGGAAAATTCTCAAAATGCTCCACTTGTACATGCAACCTTGGAAACATTGCTCAGATTTCTGAACTGGA
TTCCCCTGGGATATATTTTTGAGACCAAATTAATCAGCACATTGATTTATAAGTTCCTGAATGTTCCAAT
GTTTCGAAATGTCTCTCTGAAGTGCCTCACTGAGATTGCTGGTGTGAGTGTAAGCCAATATGAAGAACAA
TTTGTAACACTATTTACTCTGACAATGATGCAACTAAAGCAGATGCTTCCTTTAAATACCAATATTCGAC
TTGCGTACTCAAATGGAAAAGATGATGAACAGAACTTCATTCAAAATCTCAGTTTGTTTCTCTGCACCTT
TCTTAAGGAACATGATCAACTTATAGAAAAAAGATTAAATCTCAGGGAAACTCTTATGGAGGCCCTTCAT
TATATGTTGTTGGTATCTGAAGTAGAAGAAACTGAAATCTTTAAAATTTGTCTTGAATACTGGAATCATT
TGGCTGCTGAACTCTATAGAGAGAGTCCATTCTCTACATCTGCCTCTCCGTTGCTTTCTGGAAGTCAACA
TTTTGATGTTCCTCCCAGGAGACAGCTATATTTGCCCATGTTATTCAAGGTCCGTTTATTAATGGTTAGT
CGAATGGCTAAACCAGAGGAAGTATTGGTTGTAGAGAATGATCAAGGAGAAGTTGTGAGAGAATTCATGA
AGGATACAGATTCCATAAATTTGTATAAGAATATGAGGGAAACATTGGTTTATCTTACTCATCTGGATTA
TGTAGATACAGAAAGAATAATGACAGAGAAGCTTCACAATCAAGTGAATGGTACAGAGTGGTCATGGAAA
AATTTGAATACATTGTGTTGGGCAATAGGCTCCATTAGTGGAGCAATGCATGAAGAGGACGAAAAACGAT
TTCTTGTTACTGTTATAAAGGATCTATTAGGATTATGTGAACAGAAAAGAGGCAAAGATAATAAAGCTAT
TATTGCATCAAATATCATGTACATAGTAGGTCAATACCCACGTTTTTTGAGAGCTCACTGGAAATTTCTG
AAGACTGTAGTTAACAAGCTGTTCGAATTCATGCATGAGACCCATGATGGAGTCCAGGATATGGCTTGTG
ATACTTTCATTAAAATAGCCCAAAAATGCCGCAGGCATTTCGTTCAGGTTCAGGTTGGAGAAGTGATGCC
ATTTATTGATGAAATTTTGAACAACATTAACACTATTATTTGTGATCTTCAGCCTCAACAGAATGTGGAT
ATACTGAAAGATCCTGAAACAGTCAAGCAGCTTGGTAGCATTTTGAAAACAAATGTGAGAGCCTGCAAAG
CTGTTGGACACCCCTTTGTAATTCAGCTTGGAAGAATTTATTTAGATATGCTTAATGTATACAAGTGCCT
CAGTGAAAATATTTCTGCAGCTATCCAAGCTAATGGTGAAATGGTTACAAAGCAACCATTGATTAGAAGT
ATGCGAACTGTAAAAAGGGAAACTTTAAAGTTAATATCTGGTTGGGTGAGCCGATCCAATGATCCACAGA
TGGTCGCTGAAAATTTTGTTCCCCCTCTGTTGGATGCAGTTCTCATTGATTATCAGAGAAATGTCCCAGC
TGCTAGAGAACCAGAAGTGCTTAGTACTATGGCCATAATTGTCAACAAGTTAGGGGGACATATAACAGCT
GAAATACCTCAAATATTTGATGCTGTTTTTGAATGCACATTGAATATGATAAATAAGGACTTTGAAGAAT
ATCCTGAACATAGAACGAACTTTTTCTTACTACTTCAGGCTGTCAATTCTCATTGTTTCCCAGCATTCCT
TGCTATTCCACCTACACAGTTTAAACTTGTTTTGGATTCCATCATTTGGGCTTTCAAACATACTATGAGG
AATGTCGCAGATACGGGCTTACAGATACTTTTTACACTCTTACAAAATGTTGCACAAGAAGAAGCTGCAG
CTCAGAGTTTTTATCAAACTTATTTTTGTGATATTCTCCAGCATATCTTTTCTGTTGTGACAGACACTTC
ACATACTGCTGGTTTAACAATGCATGCATCAATTCTTGCATATATGTTTAATTTGGTTGAAGAAGGAAAA
ATAAGTACATCATTAAATCCTGGAAATCCAGTTAACAACCAAATCTTTCTTCAGGAATATGTGGCTAATC
TCCTTAAGTCGGCCTTCCCTCACCTACAAGATGCTCAAGTAAAGCTCTTTGTGACAGGGCTTTTCAGCTT
AAATCAAGATATTCCTGCTTTCAAGGAACATTTAAGAGATTTCCTAGTTCAAATAAAGGAATTTGCAGGT
GAAGACACTTCTGATTTGTTTTTGGAAGAGAGAGAAATAGCCCTACGGCAGGCTGATGAAGAGAAACATA
AACGTCAAATGTCTGTCCCTGGCATCTTTAATCCACATGAGATTCCAGAAGAAATGTGTGATTAAAATCC
AAATTCATGCTGTTTTTTTTCTCTGCAACTCGTTAGCAGAGGAAAACAGCATGTGGGTATTTGTCGACCA
AAATGATGCCAATTTGTAAATTAAAATGTCACCTAGTGGCCCTTTTTCTTATGTGTTTTTTTGTATAAGA
AATTTTCTGTGAAATATCCTTCCATTGTTTAAGCTTTTGTTTTGGTCATCTTTATTTAGTTTGCATGAAG
TTGAAAATTAAGGCATTTTTAAAAATTTTACTTCATGCCCATTTTTGTGGCTGGGCTGGGGGGAGGAGGC
AAATTCGATTTGAACATATACTTGTAATTCTAATGCAAAATTATACAATTTTTCCTGTAAACAATACCAA
TTTTTAATTAGGGAGCATTTTCCTTCTAGTCTATTTCAGCCTAGAAGAAAAGATAATGAGTAAAACAAAT
TGCGTTGTTTAAAGGATTATAGTGCTGCATTGTCTGAAGTTAGCACCTCTTGGACTGAATCGTTTGTCTA
GACTACATGTATTACAAAGTCTCTTTGGCAAGATTGCAGCAAGATCATGTGCATATCATCCCATTGTAAA
GCGACTTCAAAAATATGGGAACACAGTTAGTTATTTTTACACAGTTCTTTTTGTTTTTGTGTGTGTGTGC
TGTCGCTTGTCGACAACAGCTTTTTGTTTTCCTCAATGAGGAGTGTTGCTCATTTGTGAGCCTTCATTAA
CTCGAAGTGAAATGGTTAAAAATATTTATCCTGTTAGAATAGGCTGCATCTTTTTAACAACTCATTAAAA
AACAAAACAACTCTGGCTTTTGAGATGACTTATACTAATTTACATTGTTTACCAAGCTGTAGTGCTTTAA
GAACACTACTTAAAAAGCAAAATAAACTTGGTTTACATTTA
>XM_011533099.3 PREDICTED: Homo sapiens exportin 1 (XPO1),
transcript variant X7, mRNA
(SEQ ID NO: 4)
AGCGCCGGAGCCGCGTGAGAGAGGGAGCCGTGTTTTGGTAGGGGGGAGTCGGACTGCAACTGGCAGCAGA
GCGTCTCCCCGGCCGTGTGGACTCTACACCCCCTACTCCTGCCGCTTCTGCTGCTGCCTGTGGCTGGAGG
GTCCCCCTGGGGCTGAATCTTTGGGACTTGACCCCGTTCCCTCCCCCTTCCCTCACTCCCCAGCCGGGCG
GGAGCATTTATTCCCCAGATTAATTCCCCTTTTGGGGGGGGGGGGGTGTGTGTGTGTGTGTGTGTGTGTG
TGTGTGTTGGGGGAAGCGTCCCTGAAATAGTAAATATTATTGAGCTCTTTTTGCCCTTTTCCTGTCCGTT
TTTTTAATTTCCTTTTTTGAGGTGGGAAAACTGAAACCCACCTTGATTCGTCCCCTCTCCCCCCTCCCCA
CCTTCCCTCGCCCTAATCCCCCAACGAGGAAGGAAGGAGCAGTTGGTTCAATCTCTGGTAATCTATGCCA
GCAATTATGACAATGTTAGCAGACCATGCAGCTCGTCAGCTGCTTGATTTCAGCCAAAAACTGGATATCA
ACTTATTAGATAATGTGGTGAATTGCTTATACCATGGAGAAGGAGCCCAGCAAAGAATGGCTCAAGAAGT
ACTGACACATTTAAAGGAGCATCCTGATGCTTGGACAAGAGTCGACACAATTTTGGAATTTTCTCAGAAT
ATGAATACGAAAGTAAGCAAGTGGTGGTTTTAAAATCTGTTATTTCTTTTCTTCCAGTCATAAACTTTGA
GGAAGGTATCTCATTTTTAGTATTAGCATTTGTTATCTCAAATTATTTTATATTGTTAATTTGTTTCAAA
ATTTAGTCTTAAGCATATAATTAATTCTTGCAAATAGTCCTAAGAATTATGAATGAAGTTTTGTAATGGG
ACTAGTATGCAAGGTGGCTTTTTTGGTTGTCTGATTTTGTTTTTTTCTTTTTAGTATATGTATACAGGTC
TAAGTAGCATAAATTCTGAATGTTATTCAGAGATTAAACATAACATTAAGGAATTATTTTGGGCAAGTAA
TCAATATAATACAGGCCAGGCACCTCTGACACCAAGTTGTGTGGACAGCTTAAAACCCTACTCCATGGCA
TTGGGCTTCTAATGGGACAGCACTCAGTTTTTATTTACAATGGAAAATGTTATAATTCTGGTCCTTTTTT
AAGTTTGAACAGAAGGGTTGATCAAAATGTGTTTTGTCTGTTTTAGGCTTGATGATTCACGCTTCTCTTT
AAACTGCCTTAAAGTAATAAATACTATGGCATTCTGTTTAATACACGAAAGGTTTCCACTTGATATACAT
TGTGTTAAAACAGAAGATAGCCATATTCACATCTGTTTCTGAAACCAGACTTTGTTAATTAACAGTTTTA
AAATATTAATTAGTTTTGACATCTTGTAAATTTTAGGTCAAAAATACAGTCCAGAACTTCCTCAAAGTGC
TAACCTTAAGTAACATAACAGTGTTTGCCTGTTTGCTTGCCTGGTAAATTTTGCTTTTTATACTGACAGA
CATTACTAATGTTTAAGTTGATTTATCAACAGTATTTTTAAAAACTCAAATTTGTCTTGTGAAAAATGAG
ATGATTAGAGGAACAGAAAGAAAGCAACAGTACTATGGACTACAAATTTTGGAAAATGTGATAAAAACAA
GGTGGAAGATTCTTCCAAGGAACCAGTGCGAAGGAATAAAAAAATACGTTGTTGGCCTCATTATCAAGAC
GTCATCTGACCCAACTTGTGTAGAGAAAGAAAAGGTGTATATCGGAAAATTAAATATGATCCTTGTTCAG
ATACTGAAACAAGAATGGCCCAAACATTGGCCAACTTTTATCAGTGATATTGTTGGAGCAAGTAGGACCA
GCGAAAGTCTCTGTCAAAATAATATGGTGATTCTTAAACTCTTGAGTGAAGAAGTATTTGATTTCTCTAG
TGGACAGATAACCCAAGTCAAATCTAAGCATTTAAAAGACAGCATGTGCAATGAATTCTCACAGATATTT
CAACTGTGTCAGTTTGTAATGGAAAATTCTCAAAATGCTCCACTTGTACATGCAACCTTGGAAACATTGC
TCAGATTTCTGAACTGGATTCCCCTGGGATATATTTTTGAGACCAAATTAATCAGCACATTGATTTATAA
GTTCCTGAATGTTCCAATGTTTCGAAATGTCTCTCTGAAGTGCCTCACTGAGATTGCTGGTGTGAGTGTA
AGCCAATATGAAGAACAATTTGTAACACTATTTACTCTGACAATGATGCAACTAAAGCAGATGCTTCCTT
TAAATACCAATATTCGACTTGCGTACTCAAATGGAAAAGATGATGAACAGAACTTCATTCAAAATCTCAG
TTTGTTTCTCTGCACCTTTCTTAAGGAACATGATCAACTTATAGAAAAAAGATTAAATCTCAGGGAAACT
CTTATGGAGGCCCTTCATTATATGTTGTTGGTATCTGAAGTAGAAGAAACTGAAATCTTTAAAATTTGTC
TTGAATACTGGAATCATTTGGCTGCTGAACTCTATAGAGAGAGTCCATTCTCTACATCTGCCTCTCCGTT
GCTTTCTGGAAGTCAACATTTTGATGTTCCTCCCAGGAGACAGCTATATTTGCCCATGTTATTCAAGGTC
CGTTTATTAATGGTTAGTCGAATGGCTAAACCAGAGGAAGTATTGGTTGTAGAGAATGATCAAGGAGAAG
TTGTGAGAGAATTCATGAAGGATACAGATTCCATAAATTTGTATAAGAATATGAGGGAAACATTGGTTTA
TCTTACTCATCTGGATTATGTAGATACAGAAAGAATAATGACAGAGAAGCTTCACAATCAAGTGAATGGT
ACAGAGTGGTCATGGAAAAATTTGAATACATTGTGTTGGGCAATAGGCTCCATTAGTGGAGCAATGCATG
AAGAGGACGAAAAACGATTTCTTGTTACTGTTATAAAGGATCTATTAGGATTATGTGAACAGAAAAGAGG
CAAAGATAATAAAGCTATTATTGCATCAAATATCATGTACATAGTAGGTCAATACCCACGTTTTTTGAGA
GCTCACTGGAAATTTCTGAAGACTGTAGTTAACAAGCTGTTCGAATTCATGCATGAGACCCATGATGGAG
TCCAGGATATGGCTTGTGATACTTTCATTAAAATAGCCCAAAAATGCCGCAGGCATTTCGTTCAGGTTCA
GGTTGGAGAAGTGATGCCATTTATTGATGAAATTTTGAACAACATTAACACTATTATTTGTGATCTTCAG
CCTCAACAGGTTCATACGTTTTATGAAGCTGTGGGGTACATGATTGGTGCACAAACAGATCAAACAGTAC
AAGAACACTTGATAGAAAAGTACATGTTACTCCCTAATCAAGTGTGGGATAGTATAATCCAGCAGGCAAC
CAAAAATGTGGATATACTGAAAGATCCTGAAACAGTCAAGCAGCTTGGTAGCATTTTGAAAACAAATGTG
AGAGCCTGCAAAGCTGTTGGACACCCCTTTGTAATTCAGCTTGGAAGAATTTATTTAGATATGCTTAATG
TATACAAGTGCCTCAGTGAAAATATTTCTGCAGCTATCCAAGCTAATGGTGAAATGGTTACAAAGCAACC
ATTGATTAGAAGTATGCGAACTGTAAAAAGGGAAACTTTAAAGTTAATATCTGGTTGGGTGAGCCGATCC
AATGATCCACAGATGGTCGCTGAAAATTTTGTTCCCCCTCTGTTGGATGCAGTTCTCATTGATTATCAGA
GAAATGTCCCAGCTGCTAGAGAACCAGAAGTGCTTAGTACTATGGCCATAATTGTCAACAAGTTAGGGGG
ACATATAACAGCTGAAATACCTCAAATATTTGATGCTGTTTTTGAATGCACATTGAATATGATAAATAAG
GACTTTGAAGAATATCCTGAACATAGAACGAACTTTTTCTTACTACTTCAGGCTGTCAATTCTCATTGTT
TCCCAGCATTCCTTGCTATTCCACCTACACAGTTTAAACTTGTTTTGGATTCCATCATTTGGGCTTTCAA
ACATACTATGAGGAATGTCGCAGATACGGGCTTACAGATACTTTTTACACTCTTACAAAATGTTGCACAA
GAAGAAGCTGCAGCTCAGAGTTTTTATCAAACTTATTTTTGTGATATTCTCCAGCATATCTTTTCTGTTG
TGACAGACACTTCACATACTGCTGGTTTAACAATGCATGCATCAATTCTTGCATATATGTTTAATTTGGT
TGAAGAAGGAAAAATAAGTACATCATTAAATCCTGGAAATCCAGTTAACAACCAAATCTTTCTTCAGGAA
TATGTGGCTAATCTCCTTAAGTCGGCCTTCCCTCACCTACAAGATGCTCAAGTAAAGCTCTTTGTGACAG
GGCTTTTCAGCTTAAATCAAGATATTCCTGCTTTCAAGGAACATTTAAGAGATTTCCTAGTTCAAATAAA
GGAATTTGCAGGTGAAGACACTTCTGATTTGTTTTTGGAAGAGAGAGAAATAGCCCTACGGCAGGCTGAT
GAAGAGAAACATAAACGTCAAATGTCTGTCCCTGGCATCTTTAATCCACATGAGATTCCAGAAGAAATGT
GTGATTAAAATCCAAATTCATGCTGTTTTTTTTCTCTGCAACTCGTTAGCAGAGGAAAACAGCATGTGGG
TATTTGTCGACCAAAATGATGCCAATTTGTAAATTAAAATGTCACCTAGTGGCCCTTTTTCTTATGTGTT
TTTTTGTATAAGAAATTTTCTGTGAAATATCCTTCCATTGTTTAAGCTTTTGTTTTGGTCATCTTTATTT
AGTTTGCATGAAGTTGAAAATTAAGGCATTTTTAAAAATTTTACTTCATGCCCATTTTTGTGGCTGGGCT
GGGGGGAGGAGGCAAATTCGATTTGAACATATACTTGTAATTCTAATGCAAAATTATACAATTTTTCCTG
TAAACAATACCAATTTTTAATTAGGGAGCATTTTCCTTCTAGTCTATTTCAGCCTAGAAGAAAAGATAAT
GAGTAAAACAAATTGCGTTGTTTAAAGGATTATAGTGCTGCATTGTCTGAAGTTAGCACCTCTTGGACTG
AATCGTTTGTCTAGACTACATGTATTACAAAGTCTCTTTGGCAAGATTGCAGCAAGATCATGTGCATATC
ATCCCATTGTAAAGCGACTTCAAAAATATGGGAACACAGTTAGTTATTTTTACACAGTTCTTTTTGTTTT
TGTGTGTGTGTGCTGTCGCTTGTCGACAACAGCTTTTTGTTTTCCTCAATGAGGAGTGTTGCTCATTTGT
GAGCCTTCATTAACTCGAAGTGAAATGGTTAAAAATATTTATCCTGTTAGAATAGGCTGCATCTTTTTAA
CAACTCATTAAAAAACAAAACAACTCTGGCTTTTGAGATGACTTATACTAATTTACATTGTTTACCAAGC
TGTAGTGCTTTAAGAACACTACTTAAAAAGCAAAATAAACTTGGTTTACATTTA
>XM 006712094.3 PREDICTED: Homo sapiens exportin 1 (XPO1),
transcript variant X1, mRNA
(SEQ ID NO: 5)
GTCCCCTCTCCCCCCTCCCCACCTTCCCTCGCCCTAATCCCCCAACGAGGAAGGAAGGAGCAGTTGGTTC
AATCTCTGGATGGAGTCTCATTCTCTTGCCCAGGCTAGAGTGTGGTGGTGCATCTCGGCTTACTGCAAAC
TCCGTCTCCTGGGTTCAAGCAGTTCTCCTGCCTCAGCTTCCCAAGTAGCTGGGATTACAGTAATCTATGC
CAGCAATTATGACAATGTTAGCAGACCATGCAGCTCGTCAGCTGCTTGATTTCAGCCAAAAACTGGATAT
CAACTTATTAGATAATGTGGTGAATTGCTTATACCATGGAGAAGGAGCCCAGCAAAGAATGGCTCAAGAA
GTACTGACACATTTAAAGGAGCATCCTGATGCTTGGACAAGAGTCGACACAATTTTGGAATTTTCTCAGA
ATATGAATACGAAATACTATGGACTACAAATTTTGGAAAATGTGATAAAAACAAGGTGGAAGATTCTTCC
AAGGAACCAGTGCGAAGGAATAAAAAAATACGTTGTTGGCCTCATTATCAAGACGTCATCTGACCCAACT
TGTGTAGAGAAAGAAAAGGTGTATATCGGAAAATTAAATATGATCCTTGTTCAGATACTGAAACAAGAAT
GGCCCAAACATTGGCCAACTTTTATCAGTGATATTGTTGGAGCAAGTAGGACCAGCGAAAGTCTCTGTCA
AAATAATATGGTGATTCTTAAACTCTTGAGTGAAGAAGTATTTGATTTCTCTAGTGGACAGATAACCCAA
GTCAAATCTAAGCATTTAAAAGACAGCATGTGCAATGAATTCTCACAGATATTTCAACTGTGTCAGTTTG
TAATGGAAAATTCTCAAAATGCTCCACTTGTACATGCAACCTTGGAAACATTGCTCAGATTTCTGAACTG
GATTCCCCTGGGATATATTTTTGAGACCAAATTAATCAGCACATTGATTTATAAGTTCCTGAATGTTCCA
ATGTTTCGAAATGTCTCTCTGAAGTGCCTCACTGAGATTGCTGGTGTGAGTGTAAGCCAATATGAAGAAC
AATTTGTAACACTATTTACTCTGACAATGATGCAACTAAAGCAGATGCTTCCTTTAAATACCAATATTCG
ACTTGCGTACTCAAATGGAAAAGATGATGAACAGAACTTCATTCAAAATCTCAGTTTGTTTCTCTGCACC
TTTCTTAAGGAACATGATCAACTTATAGAAAAAAGATTAAATCTCAGGGAAACTCTTATGGAGGCCCTTC
ATTATATGTTGTTGGTATCTGAAGTAGAAGAAACTGAAATCTTTAAAATTTGTCTTGAATACTGGAATCA
TTTGGCTGCTGAACTCTATAGAGAGAGTCCATTCTCTACATCTGCCTCTCCGTTGCTTTCTGGAAGTCAA
CATTTTGATGTTCCTCCCAGGAGACAGCTATATTTGCCCATGTTATTCAAGGTCCGTTTATTAATGGTTA
GTCGAATGGCTAAACCAGAGGAAGTATTGGTTGTAGAGAATGATCAAGGAGAAGTTGTGAGAGAATTCAT
GAAGGATACAGATTCCATAAATTTGTATAAGAATATGAGGGAAACATTGGTTTATCTTACTCATCTGGAT
TATGTAGATACAGAAAGAATAATGACAGAGAAGCTTCACAATCAAGTGAATGGTACAGAGTGGTCATGGA
AAAATTTGAATACATTGTGTTGGGCAATAGGCTCCATTAGTGGAGCAATGCATGAAGAGGACGAAAAACG
ATTTCTTGTTACTGTTATAAAGGATCTATTAGGATTATGTGAACAGAAAAGAGGCAAAGATAATAAAGCT
ATTATTGCATCAAATATCATGTACATAGTAGGTCAATACCCACGTTTTTTGAGAGCTCACTGGAAATTTC
TGAAGACTGTAGTTAACAAGCTGTTCGAATTCATGCATGAGACCCATGATGGAGTCCAGGATATGGCTTG
TGATACTTTCATTAAAATAGCCCAAAAATGCCGCAGGCATTTCGTTCAGGTTCAGGTTGGAGAAGTGATG
CCATTTATTGATGAAATTTTGAACAACATTAACACTATTATTTGTGATCTTCAGCCTCAACAGGTTCATA
CGTTTTATGAAGCTGTGGGGTACATGATTGGTGCACAAACAGATCAAACAGTACAAGAACACTTGATAGA
AAAGTACATGTTACTCCCTAATCAAGTGTGGGATAGTATAATCCAGCAGGCAACCAAAAATGTGGATATA
CTGAAAGATCCTGAAACAGTCAAGCAGCTTGGTAGCATTTTGAAAACAAATGTGAGAGCCTGCAAAGCTG
TTGGACACCCCTTTGTAATTCAGCTTGGAAGAATTTATTTAGATATGCTTAATGTATACAAGTGCCTCAG
TGAAAATATTTCTGCAGCTATCCAAGCTAATGGTGAAATGGTTACAAAGCAACCATTGATTAGAAGTATG
CGAACTGTAAAAAGGGAAACTTTAAAGTTAATATCTGGTTGGGTGAGCCGATCCAATGATCCACAGATGG
TCGCTGAAAATTTTGTTCCCCCTCTGTTGGATGCAGTTCTCATTGATTATCAGAGAAATGTCCCAGCTGC
TAGAGAACCAGAAGTGCTTAGTACTATGGCCATAATTGTCAACAAGTTAGGGGGACATATAACAGCTGAA
ATACCTCAAATATTTGATGCTGTTTTTGAATGCACATTGAATATGATAAATAAGGACTTTGAAGAATATC
CTGAACATAGAACGAACTTTTTCTTACTACTTCAGGCTGTCAATTCTCATTGTTTCCCAGCATTCCTTGC
TATTCCACCTACACAGTTTAAACTTGTTTTGGATTCCATCATTTGGGCTTTCAAACATACTATGAGGAAT
GTCGCAGATACGGGCTTACAGATACTTTTTACACTCTTACAAAATGTTGCACAAGAAGAAGCTGCAGCTC
AGAGTTTTTATCAAACTTATTTTTGTGATATTCTCCAGCATATCTTTTCTGTTGTGACAGACACTTCACA
TACTGCTGGTTTAACAATGCATGCATCAATTCTTGCATATATGTTTAATTTGGTTGAAGAAGGAAAAATA
AGTACATCATTAAATCCTGGAAATCCAGTTAACAACCAAATCTTTCTTCAGGAATATGTGGCTAATCTCC
TTAAGTCGGCCTTCCCTCACCTACAAGATGCTCAAGTAAAGCTCTTTGTGACAGGGCTTTTCAGCTTAAA
TCAAGATATTCCTGCTTTCAAGGAACATTTAAGAGATTTCCTAGTTCAAATAAAGGAATTTGCAGGTGAA
GACACTTCTGATTTGTTTTTGGAAGAGAGAGAAATAGCCCTACGGCAGGCTGATGAAGAGAAACATAAAC
GTCAAATGTCTGTCCCTGGCATCTTTAATCCACATGAGATTCCAGAAGAAATGTGTGATTAAAATCCAAA
TTCATGCTGTTTTTTTTCTCTGCAACTCGTTAGCAGAGGAAAACAGCATGTGGGTATTTGTCGACCAAAA
TGATGCCAATTTGTAAATTAAAATGTCACCTAGTGGCCCTTTTTCTTATGTGTTTTTTTGTATAAGAAAT
TTTCTGTGAAATATCCTTCCATTGTTTAAGCTTTTGTTTTGGTCATCTTTATTTAGTTTGCATGAAGTTG
AAAATTAAGGCATTTTTAAAAATTTTACTTCATGCCCATTTTTGTGGCTGGGCTGGGGGGAGGAGGCAAA
TTCGATTTGAACATATACTTGTAATTCTAATGCAAAATTATACAATTTTTCCTGTAAACAATACCAATTT
TTAATTAGGGAGCATTTTCCTTCTAGTCTATTTCAGCCTAGAAGAAAAGATAATGAGTAAAACAAATTGC
GTTGTTTAAAGGATTATAGTGCTGCATTGTCTGAAGTTAGCACCTCTTGGACTGAATCGTTTGTCTAGAC
TACATGTATTACAAAGTCTCTTTGGCAAGATTGCAGCAAGATCATGTGCATATCATCCCATTGTAAAGCG
ACTTCAAAAATATGGGAACACAGTTAGTTATTTTTACACAGTTCTTTTTGTTTTTGTGTGTGTGTGCTGT
CGCTTGTCGACAACAGCTTTTTGTTTTCCTCAATGAGGAGTGTTGCTCATTTGTGAGCCTTCATTAACTC
GAAGTGAAATGGTTAAAAATATTTATCCTGTTAGAATAGGCTGCATCTTTTTAACAACTCATTAAAAAAC
AAAACAACTCTGGCTTTTGAGATGACTTATACTAATTTACATTGTTTACCAAGCTGTAGTGCTTTAAGAA
CACTACTTAAAAAGCAAAATAAACTTGGTTTACATTTA
>XM_024453125.1 PREDICTED: Homo sapiens exportin 1 (XPO1),
transcript variant X2, mRNA
(SEQ ID NO: 6)
ACATCTCTGTAAATTATTAGGAGTGGAAGTTTCCAGAAATATTTAGTGCAGCAGCATTGAAACGTCCGCC
TGCATCACGTGGCCCCCTCGGTGGAGAGCTTGGGATGGGGGAGGGAGGGGCTGCCGACTCCCTGTTCCTC
CAGCAAGGAAACTCGAAAGCTCTCCGGTTTTGCAGAGTGGCATGTGAAGTAGGAAAGATCAGTTTTTCAG
GGGCCTGCACATGGGATTCCGTCTTGTAGTTCCTTTTCAGGGGTTTGAATGTGATAAGTTGGTTAAACCA
TTTACTGTTGGCCTTGTACAGTGGAGGATGCCCTGATTCTCCTTCATTCCGATGTCTGTGAGTTTGGGTT
GGGTGGGAGGAAGTGGTGTAGTAGAGGAAGCATTCAGTACTGGCTCAGCCCGGACCTCCTCTGATCACGC
CGAGGCCATGCTGCATAGAGAAAGGGGCGGTGAACTTAAGGCGCCTGGTCTAAATGCCTCCTCCGAGTAA
GGGCGGGTAATCTATGCCAGCAATTATGACAATGTTAGCAGACCATGCAGCTCGTCAGCTGCTTGATTTC
AGCCAAAAACTGGATATCAACTTATTAGATAATGTGGTGAATTGCTTATACCATGGAGAAGGAGCCCAGC
AAAGAATGGCTCAAGAAGTACTGACACATTTAAAGGAGCATCCTGATGCTTGGACAAGAGTCGACACAAT
TTTGGAATTTTCTCAGAATATGAATACGAAATACTATGGACTACAAATTTTGGAAAATGTGATAAAAACA
AGGTGGAAGATTCTTCCAAGGAACCAGTGCGAAGGAATAAAAAAATACGTTGTTGGCCTCATTATCAAGA
CGTCATCTGACCCAACTTGTGTAGAGAAAGAAAAGGTGTATATCGGAAAATTAAATATGATCCTTGTTCA
GATACTGAAACAAGAATGGCCCAAACATTGGCCAACTTTTATCAGTGATATTGTTGGAGCAAGTAGGACC
AGCGAAAGTCTCTGTCAAAATAATATGGTGATTCTTAAACTCTTGAGTGAAGAAGTATTTGATTTCTCTA
GTGGACAGATAACCCAAGTCAAATCTAAGCATTTAAAAGACAGCATGTGCAATGAATTCTCACAGATATT
TCAACTGTGTCAGTTTGTAATGGAAAATTCTCAAAATGCTCCACTTGTACATGCAACCTTGGAAACATTG
CTCAGATTTCTGAACTGGATTCCCCTGGGATATATTTTTGAGACCAAATTAATCAGCACATTGATTTATA
AGTTCCTGAATGTTCCAATGTTTCGAAATGTCTCTCTGAAGTGCCTCACTGAGATTGCTGGTGTGAGTGT
AAGCCAATATGAAGAACAATTTGTAACACTATTTACTCTGACAATGATGCAACTAAAGCAGATGCTTCCT
TTAAATACCAATATTCGACTTGCGTACTCAAATGGAAAAGATGATGAACAGAACTTCATTCAAAATCTCA
GTTTGTTTCTCTGCACCTTTCTTAAGGAACATGATCAACTTATAGAAAAAAGATTAAATCTCAGGGAAAC
TCTTATGGAGGCCCTTCATTATATGTTGTTGGTATCTGAAGTAGAAGAAACTGAAATCTTTAAAATTTGT
CTTGAATACTGGAATCATTTGGCTGCTGAACTCTATAGAGAGAGTCCATTCTCTACATCTGCCTCTCCGT
TGCTTTCTGGAAGTCAACATTTTGATGTTCCTCCCAGGAGACAGCTATATTTGCCCATGTTATTCAAGGT
CCGTTTATTAATGGTTAGTCGAATGGCTAAACCAGAGGAAGTATTGGTTGTAGAGAATGATCAAGGAGAA
GTTGTGAGAGAATTCATGAAGGATACAGATTCCATAAATTTGTATAAGAATATGAGGGAAACATTGGTTT
ATCTTACTCATCTGGATTATGTAGATACAGAAAGAATAATGACAGAGAAGCTTCACAATCAAGTGAATGG
TACAGAGTGGTCATGGAAAAATTTGAATACATTGTGTTGGGCAATAGGCTCCATTAGTGGAGCAATGCAT
GAAGAGGACGAAAAACGATTTCTTGTTACTGTTATAAAGGATCTATTAGGATTATGTGAACAGAAAAGAG
GCAAAGATAATAAAGCTATTATTGCATCAAATATCATGTACATAGTAGGTCAATACCCACGTTTTTTGAG
AGCTCACTGGAAATTTCTGAAGACTGTAGTTAACAAGCTGTTCGAATTCATGCATGAGACCCATGATGGA
GTCCAGGATATGGCTTGTGATACTTTCATTAAAATAGCCCAAAAATGCCGCAGGCATTTCGTTCAGGTTC
AGGTTGGAGAAGTGATGCCATTTATTGATGAAATTTTGAACAACATTAACACTATTATTTGTGATCTTCA
GCCTCAACAGGTTCATACGTTTTATGAAGCTGTGGGGTACATGATTGGTGCACAAACAGATCAAACAGTA
CAAGAACACTTGATAGAAAAGTACATGTTACTCCCTAATCAAGTGTGGGATAGTATAATCCAGCAGGCAA
CCAAAAATGTGGATATACTGAAAGATCCTGAAACAGTCAAGCAGCTTGGTAGCATTTTGAAAACAAATGT
GAGAGCCTGCAAAGCTGTTGGACACCCCTTTGTAATTCAGCTTGGAAGAATTTATTTAGATATGCTTAAT
GTATACAAGTGCCTCAGTGAAAATATTTCTGCAGCTATCCAAGCTAATGGTGAAATGGTTACAAAGCAAC
CATTGATTAGAAGTATGCGAACTGTAAAAAGGGAAACTTTAAAGTTAATATCTGGTTGGGTGAGCCGATC
CAATGATCCACAGATGGTCGCTGAAAATTTTGTTCCCCCTCTGTTGGATGCAGTTCTCATTGATTATCAG
AGAAATGTCCCAGCTGCTAGAGAACCAGAAGTGCTTAGTACTATGGCCATAATTGTCAACAAGTTAGGGG
GACATATAACAGCTGAAATACCTCAAATATTTGATGCTGTTTTTGAATGCACATTGAATATGATAAATAA
GGACTTTGAAGAATATCCTGAACATAGAACGAACTTTTTCTTACTACTTCAGGCTGTCAATTCTCATTGT
TTCCCAGCATTCCTTGCTATTCCACCTACACAGTTTAAACTTGTTTTGGATTCCATCATTTGGGCTTTCA
AACATACTATGAGGAATGTCGCAGATACGGGCTTACAGATACTTTTTACACTCTTACAAAATGTTGCACA
AGAAGAAGCTGCAGCTCAGAGTTTTTATCAAACTTATTTTTGTGATATTCTCCAGCATATCTTTTCTGTT
GTGACAGACACTTCACATACTGCTGGTTTAACAATGCATGCATCAATTCTTGCATATATGTTTAATTTGG
TTGAAGAAGGAAAAATAAGTACATCATTAAATCCTGGAAATCCAGTTAACAACCAAATCTTTCTTCAGGA
ATATGTGGCTAATCTCCTTAAGTCGGCCTTCCCTCACCTACAAGATGCTCAAGTAAAGCTCTTTGTGACA
GGGCTTTTCAGCTTAAATCAAGATATTCCTGCTTTCAAGGAACATTTAAGAGATTTCCTAGTTCAAATAA
AGGAATTTGCAGGTGAAGACACTTCTGATTTGTTTTTGGAAGAGAGAGAAATAGCCCTACGGCAGGCTGA
TGAAGAGAAACATAAACGTCAAATGTCTGTCCCTGGCATCTTTAATCCACATGAGATTCCAGAAGAAATG
TGTGATTAAAATCCAAATTCATGCTGTTTTTTTTCTCTGCAACTCGTTAGCAGAGGAAAACAGCATGTGG
GTATTTGTCGACCAAAATGATGCCAATTTGTAAATTAAAATGTCACCTAGTGGCCCTTTTTCTTATGTGT
TTTTTTGTATAAGAAATTTTCTGTGAAATATCCTTCCATTGTTTAAGCTTTTGTTTTGGTCATCTTTATT
TAGTTTGCATGAAGTTGAAAATTAAGGCATTTTTAAAAATTTTACTTCATGCCCATTTTTGTGGCTGGGC
TGGGGGGAGGAGGCAAATTCGATTTGAACATATACTTGTAATTCTAATGCAAAATTATACAATTTTTCCT
GTAAACAATACCAATTTTTAATTAGGGAGCATTTTCCTTCTAGTCTATTTCAGCCTAGAAGAAAAGATAA
TGAGTAAAACAAATTGCGTTGTTTAAAGGATTATAGTGCTGCATTGTCTGAAGTTAGCACCTCTTGGACT
GAATCGTTTGTCTAGACTACATGTATTACAAAGTCTCTTTGGCAAGATTGCAGCAAGATCATGTGCATAT
CATCCCATTGTAAAGCGACTTCAAAAATATGGGAACACAGTTAGTTATTTTTACACAGTTCTTTTTGTTT
TTGTGTGTGTGTGCTGTCGCTTGTCGACAACAGCTTTTTGTTTTCCTCAATGAGGAGTGTTGCTCATTTG
TGAGCCTTCATTAACTCGAAGTGAAATGGTTAAAAATATTTATCCTGTTAGAATAGGCTGCATCTTTTTA
ACAACTCATTAAAAAACAAAACAACTCTGGCTTTTGAGATGACTTATACTAATTTACATTGTTTACCAAG
CTGTAGTGCTTTAAGAACACTACTTAAAAAGCAAAATAAACTTGGTTTACATTTA
>XM_024453126.1 PREDICTED: Homo sapiens exportin 1 (XPO1),
transcript variant X3, mRNA
(SEQ ID NO: 7)
CGGGGTTTTGCCATGTTGGCCAGGCTGGTCTGGAACTCCTGACCTCAAGTGATTCGCCCACCTCGGCCTC
CCAAAGTGCTGGGATTACAGTAATCTATGCCAGCAATTATGACAATGTTAGCAGACCATGCAGCTCGTCA
GCTGCTTGATTTCAGCCAAAAACTGGATATCAACTTATTAGATAATGTGGTGAATTGCTTATACCATGGA
GAAGGAGCCCAGCAAAGAATGGCTCAAGAAGTACTGACACATTTAAAGGAGCATCCTGATGCTTGGACAA
GAGTCGACACAATTTTGGAATTTTCTCAGAATATGAATACGAAATACTATGGACTACAAATTTTGGAAAA
TGTGATAAAAACAAGGTGGAAGATTCTTCCAAGGAACCAGTGCGAAGGAATAAAAAAATACGTTGTTGGC
CTCATTATCAAGACGTCATCTGACCCAACTTGTGTAGAGAAAGAAAAGGTGTATATCGGAAAATTAAATA
TGATCCTTGTTCAGATACTGAAACAAGAATGGCCCAAACATTGGCCAACTTTTATCAGTGATATTGTTGG
AGCAAGTAGGACCAGCGAAAGTCTCTGTCAAAATAATATGGTGATTCTTAAACTCTTGAGTGAAGAAGTA
TTTGATTTCTCTAGTGGACAGATAACCCAAGTCAAATCTAAGCATTTAAAAGACAGCATGTGCAATGAAT
TCTCACAGATATTTCAACTGTGTCAGTTTGTAATGGAAAATTCTCAAAATGCTCCACTTGTACATGCAAC
CTTGGAAACATTGCTCAGATTTCTGAACTGGATTCCCCTGGGATATATTTTTGAGACCAAATTAATCAGC
ACATTGATTTATAAGTTCCTGAATGTTCCAATGTTTCGAAATGTCTCTCTGAAGTGCCTCACTGAGATTG
CTGGTGTGAGTGTAAGCCAATATGAAGAACAATTTGTAACACTATTTACTCTGACAATGATGCAACTAAA
GCAGATGCTTCCTTTAAATACCAATATTCGACTTGCGTACTCAAATGGAAAAGATGATGAACAGAACTTC
ATTCAAAATCTCAGTTTGTTTCTCTGCACCTTTCTTAAGGAACATGATCAACTTATAGAAAAAAGATTAA
ATCTCAGGGAAACTCTTATGGAGGCCCTTCATTATATGTTGTTGGTATCTGAAGTAGAAGAAACTGAAAT
CTTTAAAATTTGTCTTGAATACTGGAATCATTTGGCTGCTGAACTCTATAGAGAGAGTCCATTCTCTACA
TCTGCCTCTCCGTTGCTTTCTGGAAGTCAACATTTTGATGTTCCTCCCAGGAGACAGCTATATTTGCCCA
TGTTATTCAAGGTCCGTTTATTAATGGTTAGTCGAATGGCTAAACCAGAGGAAGTATTGGTTGTAGAGAA
TGATCAAGGAGAAGTTGTGAGAGAATTCATGAAGGATACAGATTCCATAAATTTGTATAAGAATATGAGG
GAAACATTGGTTTATCTTACTCATCTGGATTATGTAGATACAGAAAGAATAATGACAGAGAAGCTTCACA
ATCAAGTGAATGGTACAGAGTGGTCATGGAAAAATTTGAATACATTGTGTTGGGCAATAGGCTCCATTAG
TGGAGCAATGCATGAAGAGGACGAAAAACGATTTCTTGTTACTGTTATAAAGGATCTATTAGGATTATGT
GAACAGAAAAGAGGCAAAGATAATAAAGCTATTATTGCATCAAATATCATGTACATAGTAGGTCAATACC
CACGTTTTTTGAGAGCTCACTGGAAATTTCTGAAGACTGTAGTTAACAAGCTGTTCGAATTCATGCATGA
GACCCATGATGGAGTCCAGGATATGGCTTGTGATACTTTCATTAAAATAGCCCAAAAATGCCGCAGGCAT
TTCGTTCAGGTTCAGGTTGGAGAAGTGATGCCATTTATTGATGAAATTTTGAACAACATTAACACTATTA
TTTGTGATCTTCAGCCTCAACAGGTTCATACGTTTTATGAAGCTGTGGGGTACATGATTGGTGCACAAAC
AGATCAAACAGTACAAGAACACTTGATAGAAAAGTACATGTTACTCCCTAATCAAGTGTGGGATAGTATA
ATCCAGCAGGCAACCAAAAATGTGGATATACTGAAAGATCCTGAAACAGTCAAGCAGCTTGGTAGCATTT
TGAAAACAAATGTGAGAGCCTGCAAAGCTGTTGGACACCCCTTTGTAATTCAGCTTGGAAGAATTTATTT
AGATATGCTTAATGTATACAAGTGCCTCAGTGAAAATATTTCTGCAGCTATCCAAGCTAATGGTGAAATG
GTTACAAAGCAACCATTGATTAGAAGTATGCGAACTGTAAAAAGGGAAACTTTAAAGTTAATATCTGGTT
GGGTGAGCCGATCCAATGATCCACAGATGGTCGCTGAAAATTTTGTTCCCCCTCTGTTGGATGCAGTTCT
CATTGATTATCAGAGAAATGTCCCAGCTGCTAGAGAACCAGAAGTGCTTAGTACTATGGCCATAATTGTC
AACAAGTTAGGGGGACATATAACAGCTGAAATACCTCAAATATTTGATGCTGTTTTTGAATGCACATTGA
ATATGATAAATAAGGACTTTGAAGAATATCCTGAACATAGAACGAACTTTTTCTTACTACTTCAGGCTGT
CAATTCTCATTGTTTCCCAGCATTCCTTGCTATTCCACCTACACAGTTTAAACTTGTTTTGGATTCCATC
ATTTGGGCTTTCAAACATACTATGAGGAATGTCGCAGATACGGGCTTACAGATACTTTTTACACTCTTAC
AAAATGTTGCACAAGAAGAAGCTGCAGCTCAGAGTTTTTATCAAACTTATTTTTGTGATATTCTCCAGCA
TATCTTTTCTGTTGTGACAGACACTTCACATACTGCTGGTTTAACAATGCATGCATCAATTCTTGCATAT
ATGTTTAATTTGGTTGAAGAAGGAAAAATAAGTACATCATTAAATCCTGGAAATCCAGTTAACAACCAAA
TCTTTCTTCAGGAATATGTGGCTAATCTCCTTAAGTCGGCCTTCCCTCACCTACAAGATGCTCAAGTAAA
GCTCTTTGTGACAGGGCTTTTCAGCTTAAATCAAGATATTCCTGCTTTCAAGGAACATTTAAGAGATTTC
CTAGTTCAAATAAAGGAATTTGCAGGTGAAGACACTTCTGATTTGTTTTTGGAAGAGAGAGAAATAGCCC
TACGGCAGGCTGATGAAGAGAAACATAAACGTCAAATGTCTGTCCCTGGCATCTTTAATCCACATGAGAT
TCCAGAAGAAATGTGTGATTAAAATCCAAATTCATGCTGTTTTTTTTCTCTGCAACTCGTTAGCAGAGGA
AAACAGCATGTGGGTATTTGTCGACCAAAATGATGCCAATTTGTAAATTAAAATGTCACCTAGTGGCCCT
TTTTCTTATGTGTTTTTTTGTATAAGAAATTTTCTGTGAAATATCCTTCCATTGTTTAAGCTTTTGTTTT
GGTCATCTTTATTTAGTTTGCATGAAGTTGAAAATTAAGGCATTTTTAAAAATTTTACTTCATGCCCATT
TTTGTGGCTGGGCTGGGGGGAGGAGGCAAATTCGATTTGAACATATACTTGTAATTCTAATGCAAAATTA
TACAATTTTTCCTGTAAACAATACCAATTTTTAATTAGGGAGCATTTTCCTTCTAGTCTATTTCAGCCTA
GAAGAAAAGATAATGAGTAAAACAAATTGCGTTGTTTAAAGGATTATAGTGCTGCATTGTCTGAAGTTAG
CACCTCTTGGACTGAATCGTTTGTCTAGACTACATGTATTACAAAGTCTCTTTGGCAAGATTGCAGCAAG
ATCATGTGCATATCATCCCATTGTAAAGCGACTTCAAAAATATGGGAACACAGTTAGTTATTTTTACACA
GTTCTTTTTGTTTTTGTGTGTGTGTGCTGTCGCTTGTCGACAACAGCTTTTTGTTTTCCTCAATGAGGAG
TGTTGCTCATTTGTGAGCCTTCATTAACTCGAAGTGAAATGGTTAAAAATATTTATCCTGTTAGAATAGG
CTGCATCTTTTTAACAACTCATTAAAAAACAAAACAACTCTGGCTTTTGAGATGACTTATACTAATTTAC
ATTGTTTACCAAGCTGTAGTGCTTTAAGAACACTACTTAAAAAGCAAAATAAACTTGGTTTACATTTA
>XM_024453127.1 PREDICTED: Homo sapiens exportin 1 (XPO1),
transcript variant X8, mRNA
(SEQ ID NO: 8)
GCCAAAAACTGGATATCAACTTATTAGATAATGTGGTGAATTGCTTATACCATGGAGAAGGAGCCCAGCA
AAGAATGGCTCAAGAAGTACTGACACATTTAAAGGAGCATCCTGATGCTTGGACAAGAGTCGACACAATT
TTGGAATTTTCTCAGAATATGAATACGAAAGAAGAAAAGGGGTGTCTTCCTGTGCAACTGACACAGCTAG
GCTTTGACGGCAGGCCTCCACAAGTACTATGGACTACAAATTTTGGAAAATGTGATAAAAACAAGGTGGA
AGATTCTTCCAAGGAACCAGTGCGAAGGAATAAAAAAATACGTTGTTGGCCTCATTATCAAGACGTCATC
TGACCCAACTTGTGTAGAGAAAGAAAAGGTGTATATCGGAAAATTAAATATGATCCTTGTTCAGATACTG
AAACAAGAATGGCCCAAACATTGGCCAACTTTTATCAGTGATATTGTTGGAGCAAGTAGGACCAGCGAAA
GTCTCTGTCAAAATAATATGGTGATTCTTAAACTCTTGAGTGAAGAAGTATTTGATTTCTCTAGTGGACA
GATAACCCAAGTCAAATCTAAGCATTTAAAAGACAGCATGTGCAATGAATTCTCACAGATATTTCAACTG
TGTCAGTTTGTAATGGAAAATTCTCAAAATGCTCCACTTGTACATGCAACCTTGGAAACATTGCTCAGAT
TTCTGAACTGGATTCCCCTGGGATATATTTTTGAGACCAAATTAATCAGCACATTGATTTATAAGTTCCT
GAATGTTCCAATGTTTCGAAATGTCTCTCTGAAGTGCCTCACTGAGATTGCTGGTGTGAGTGTAAGCCAA
TATGAAGAACAATTTGTAACACTATTTACTCTGACAATGATGCAACTAAAGCAGATGCTTCCTTTAAATA
CCAATATTCGACTTGCGTACTCAAATGGAAAAGATGATGAACAGAACTTCATTCAAAATCTCAGTTTGTT
TCTCTGCACCTTTCTTAAGGAACATGATCAACTTATAGAAAAAAGATTAAATCTCAGGGAAACTCTTATG
GAGGCCCTTCATTATATGTTGTTGGTATCTGAAGTAGAAGAAACTGAAATCTTTAAAATTTGTCTTGAAT
ACTGGAATCATTTGGCTGCTGAACTCTATAGAGAGAGTCCATTCTCTACATCTGCCTCTCCGTTGCTTTC
TGGAAGTCAACATTTTGATGTTCCTCCCAGGAGACAGCTATATTTGCCCATGTTATTCAAGGTCCGTTTA
TTAATGGTTAGTCGAATGGCTAAACCAGAGGAAGTATTGGTTGTAGAGAATGATCAAGGAGAAGTTGTGA
GAGAATTCATGAAGGATACAGATTCCATAAATTTGTATAAGAATATGAGGGAAACATTGGTTTATCTTAC
TCATCTGGATTATGTAGATACAGAAAGAATAATGACAGAGAAGCTTCACAATCAAGTGAATGGTACAGAG
TGGTCATGGAAAAATTTGAATACATTGTGTTGGGCAATAGGCTCCATTAGTGGAGCAATGCATGAAGAGG
ACGAAAAACGATTTCTTGTTACTGTTATAAAGGATCTATTAGGATTATGTGAACAGAAAAGAGGCAAAGA
TAATAAAGCTATTATTGCATCAAATATCATGTACATAGTAGGTCAATACCCACGTTTTTTGAGAGCTCAC
TGGAAATTTCTGAAGACTGTAGTTAACAAGCTGTTCGAATTCATGCATGAGACCCATGATGGAGTCCAGG
ATATGGCTTGTGATACTTTCATTAAAATAGCCCAAAAATGCCGCAGGCATTTCGTTCAGGTTCAGGTTGG
AGAAGTGATGCCATTTATTGATGAAATTTTGAACAACATTAACACTATTATTTGTGATCTTCAGCCTCAA
CAGGTTCATACGTTTTATGAAGCTGTGGGGTACATGATTGGTGCACAAACAGATCAAACAGTACAAGAAC
ACTTGATAGAAAAGTACATGTTACTCCCTAATCAAGTGTGGGATAGTATAATCCAGCAGGCAACCAAAAA
TGTGGATATACTGAAAGATCCTGAAACAGTCAAGCAGCTTGGTAGCATTTTGAAAACAAATGTGAGAGCC
TGCAAAGCTGTTGGACACCCCTTTGTAATTCAGCTTGGAAGAATTTATTTAGATATGCTTAATGTATACA
AGTGCCTCAGTGAAAATATTTCTGCAGCTATCCAAGCTAATGGTGAAATGGTTACAAAGCAACCATTGAT
TAGAAGTATGCGAACTGTAAAAAGGGAAACTTTAAAGTTAATATCTGGTTGGGTGAGCCGATCCAATGAT
CCACAGATGGTCGCTGAAAATTTTGTTCCCCCTCTGTTGGATGCAGTTCTCATTGATTATCAGAGAAATG
TCCCAGCTGCTAGAGAACCAGAAGTGCTTAGTACTATGGCCATAATTGTCAACAAGTTAGGGGGACATAT
AACAGCTGAAATACCTCAAATATTTGATGCTGTTTTTGAATGCACATTGAATATGATAAATAAGGACTTT
GAAGAATATCCTGAACATAGAACGAACTTTTTCTTACTACTTCAGGCTGTCAATTCTCATTGTTTCCCAG
CATTCCTTGCTATTCCACCTACACAGTTTAAACTTGTTTTGGATTCCATCATTTGGGCTTTCAAACATAC
TATGAGGAATGTCGCAGATACGGGCTTACAGATACTTTTTACACTCTTACAAAATGTTGCACAAGAAGAA
GCTGCAGCTCAGAGTTTTTATCAAACTTATTTTTGTGATATTCTCCAGCATATCTTTTCTGTTGTGACAG
ACACTTCACATACTGCTGGTTTAACAATGCATGCATCAATTCTTGCATATATGTTTAATTTGGTTGAAGA
AGGAAAAATAAGTACATCATTAAATCCTGGAAATCCAGTTAACAACCAAATCTTTCTTCAGGAATATGTG
GCTAATCTCCTTAAGTCGGCCTTCCCTCACCTACAAGATGCTCAAGTAAAGCTCTTTGTGACAGGGCTTT
TCAGCTTAAATCAAGATATTCCTGCTTTCAAGGAACATTTAAGAGATTTCCTAGTTCAAATAAAGGAATT
TGCAGGTGAAGACACTTCTGATTTGTTTTTGGAAGAGAGAGAAATAGCCCTACGGCAGGCTGATGAAGAG
AAACATAAACGTCAAATGTCTGTCCCTGGCATCTTTAATCCACATGAGATTCCAGAAGAAATGTGTGATT
AAAATCCAAATTCATGCTGTTTTTTTTCTCTGCAACTCGTTAGCAGAGGAAAACAGCATGTGGGTATTTG
TCGACCAAAATGATGCCAATTTGTAAATTAAAATGTCACCTAGTGGCCCTTTTTCTTATGTGTTTTTTTG
TATAAGAAATTTTCTGTGAAATATCCTTCCATTGTTTAAGCTTTTGTTTTGGTCATCTTTATTTAGTTTG
CATGAAGTTGAAAATTAAGGCATTTTTAAAAATTTTACTTCATGCCCATTTTTGTGGCTGGGCTGGGGGG
AGGAGGCAAATTCGATTTGAACATATACTTGTAATTCTAATGCAAAATTATACAATTTTTCCTGTAAACA
ATACCAATTTTTAATTAGGGAGCATTTTCCTTCTAGTCTATTTCAGCCTAGAAGAAAAGATAATGAGTAA
AACAAATTGCGTTGTTTAAAGGATTATAGTGCTGCATTGTCTGAAGTTAGCACCTCTTGGACTGAATCGT
TTGTCTAGACTACATGTATTACAAAGTCTCTTTGGCAAGATTGCAGCAAGATCATGTGCATATCATCCCA
TTGTAAAGCGACTTCAAAAATATGGGAACACAGTTAGTTATTTTTACACAGTTCTTTTTGTTTTTGTGTG
TGTGTGCTGTCGCTTGTCGACAACAGCTTTTTGTTTTCCTCAATGAGGAGTGTTGCTCATTTGTGAGCCT
TCATTAACTCGAAGTGAAATGGTTAAAAATATTTATCCTGTTAGAATAGGCTGCATCTTTTTAACAACTC
ATTAAAAAACAAAACAACTCTGGCTTTTGAGATGACTTATACTAATTTACATTGTTTACCAAGCTGTAGT
GCTTTAAGAACACTACTTAAAAAGCAAAATAAACTTGGTTTACATTTA
>XM_005264546.2 PREDICTED: Homo sapiens exportin 1 (XPO1),
transcript variant X9, mRNA
(SEQ ID NO: 9)
GAAAGAAAGCAACAGGAAGAAAAGGGGTGTCTTCCTGTGCAACTGACACAGCTAGGCTTTGACGGCAGGC
CTCCACAAGTACTATGGACTACAAATTTTGGAAAATGTGATAAAAACAAGGTGGAAGATTCTTCCAAGGA
ACCAGTGCGAAGGAATAAAAAAATACGTTGTTGGCCTCATTATCAAGACGTCATCTGACCCAACTTGTGT
AGAGAAAGAAAAGGTGTATATCGGAAAATTAAATATGATCCTTGTTCAGATACTGAAACAAGAATGGCCC
AAACATTGGCCAACTTTTATCAGTGATATTGTTGGAGCAAGTAGGACCAGCGAAAGTCTCTGTCAAAATA
ATATGGTGATTCTTAAACTCTTGAGTGAAGAAGTATTTGATTTCTCTAGTGGACAGATAACCCAAGTCAA
ATCTAAGCATTTAAAAGACAGCATGTGCAATGAATTCTCACAGATATTTCAACTGTGTCAGTTTGTAATG
GAAAATTCTCAAAATGCTCCACTTGTACATGCAACCTTGGAAACATTGCTCAGATTTCTGAACTGGATTC
CCCTGGGATATATTTTTGAGACCAAATTAATCAGCACATTGATTTATAAGTTCCTGAATGTTCCAATGTT
TCGAAATGTCTCTCTGAAGTGCCTCACTGAGATTGCTGGTGTGAGTGTAAGCCAATATGAAGAACAATTT
GTAACACTATTTACTCTGACAATGATGCAACTAAAGCAGATGCTTCCTTTAAATACCAATATTCGACTTG
CGTACTCAAATGGAAAAGATGATGAACAGAACTTCATTCAAAATCTCAGTTTGTTTCTCTGCACCTTTCT
TAAGGAACATGATCAACTTATAGAAAAAAGATTAAATCTCAGGGAAACTCTTATGGAGGCCCTTCATTAT
ATGTTGTTGGTATCTGAAGTAGAAGAAACTGAAATCTTTAAAATTTGTCTTGAATACTGGAATCATTTGG
CTGCTGAACTCTATAGAGAGAGTCCATTCTCTACATCTGCCTCTCCGTTGCTTTCTGGAAGTCAACATTT
TGATGTTCCTCCCAGGAGACAGCTATATTTGCCCATGTTATTCAAGGTCCGTTTATTAATGGTTAGTCGA
ATGGCTAAACCAGAGGAAGTATTGGTTGTAGAGAATGATCAAGGAGAAGTIGTGAGAGAATTCATGAAGG
ATACAGATTCCATAAATTTGTATAAGAATATGAGGGAAACATTGGTTTATCTTACTCATCTGGATTATGT
AGATACAGAAAGAATAATGACAGAGAAGCTTCACAATCAAGTGAATGGTACAGAGTGGTCATGGAAAAAT
TTGAATACATTGTGTTGGGCAATAGGCTCCATTAGTGGAGCAATGCATGAAGAGGACGAAAAACGATTTC
TTGTTACTGTTATAAAGGATCTATTAGGATTATGTGAACAGAAAAGAGGCAAAGATAATAAAGCTATTAT
TGCATCAAATATCATGTACATAGTAGGTCAATACCCACGTTTTTTGAGAGCTCACTGGAAATTTCTGAAG
ACTGTAGTTAACAAGCTGTTCGAATTCATGCATGAGACCCATGATGGAGTCCAGGATATGGCTTGTGATA
CTTTCATTAAAATAGCCCAAAAATGCCGCAGGCATTTCGTTCAGGTTCAGGTTGGAGAAGTGATGCCATT
TATTGATGAAATTTTGAACAACATTAACACTATTATTTGTGATCTTCAGCCTCAACAGGTTCATACGTTT
TATGAAGCTGTGGGGTACATGATTGGTGCACAAACAGATCAAACAGTACAAGAACACTTGATAGAAAAGT
ACATGTTACTCCCTAATCAAGTGTGGGATAGTATAATCCAGCAGGCAACCAAAAATGTGGATATACTGAA
AGATCCTGAAACAGTCAAGCAGCTTGGTAGCATTTTGAAAACAAATGTGAGAGCCTGCAAAGCTGTTGGA
CACCCCTTTGTAATTCAGCTTGGAAGAATTTATTTAGATATGCTTAATGTATACAAGTGCCTCAGTGAAA
ATATTTCTGCAGCTATCCAAGCTAATGGTGAAATGGTTACAAAGCAACCATTGATTAGAAGTATGCGAAC
TGTAAAAAGGGAAACTTTAAAGTTAATATCTGGTTGGGTGAGCCGATCCAATGATCCACAGATGGTCGCT
GAAAATTTTGTTCCCCCTCTGTTGGATGCAGTTCTCATTGATTATCAGAGAAATGTCCCAGCTGCTAGAG
AACCAGAAGTGCTTAGTACTATGGCCATAATTGTCAACAAGTTAGGGGGACATATAACAGCTGAAATACC
TCAAATATTTGATGCTGTTTTTGAATGCACATTGAATATGATAAATAAGGACTTTGAAGAATATCCTGAA
CATAGAACGAACTTTTTCTTACTACTTCAGGCTGTCAATTCTCATTGTTTCCCAGCATTCCTTGCTATTC
CACCTACACAGTTTAAACTTGTTTTGGATTCCATCATTTGGGCTTTCAAACATACTATGAGGAATGTCGC
AGATACGGGCTTACAGATACTTTTTACACTCTTACAAAATGTTGCACAAGAAGAAGCTGCAGCTCAGAGT
TTTTATCAAACTTATTTTTGTGATATTCTCCAGCATATCTTTTCTGTTGTGACAGACACTTCACATACTG
CTGGTTTAACAATGCATGCATCAATTCTTGCATATATGTTTAATTTGGTTGAAGAAGGAAAAATAAGTAC
ATCATTAAATCCTGGAAATCCAGTTAACAACCAAATCTTTCTTCAGGAATATGTGGCTAATCTCCTTAAG
TCGGCCTTCCCTCACCTACAAGATGCTCAAGTAAAGCTCTTTGTGACAGGGCTTTTCAGCTTAAATCAAG
ATATTCCTGCTTTCAAGGAACATTTAAGAGATTTCCTAGTTCAAATAAAGGAATTTGCAGGTGAAGACAC
TTCTGATTTGTTTTTGGAAGAGAGAGAAATAGCCCTACGGCAGGCTGATGAAGAGAAACATAAACGTCAA
ATGTCTGTCCCTGGCATCTTTAATCCACATGAGATTCCAGAAGAAATGTGTGATTAAAATCCAAATTCAT
GCTGTTTTTTTTCTCTGCAACTCGTTAGCAGAGGAAAACAGCATGTGGGTATTTGTCGACCAAAATGATG
CCAATTTGTAAATTAAAATGTCACCTAGTGGCCCTTTTTCTTATGTGTTTTTTTGTATAAGAAATTTTCT
GTGAAATATCCTTCCATTGTTTAAGCTTTTGTTTTGGTCATCTTTATTTAGTTTGCATGAAGTTGAAAAT
TAAGGCATTTTTAAAAATTTTACTTCATGCCCATTTTTGTGGCTGGGCTGGGGGGAGGAGGCAAATTCGA
TTTGAACATATACTTGTAATTCTAATGCAAAATTATACAATTTTTCCTGTAAACAATACCAATTTTTAAT
TAGGGAGCATTTTCCTTCTAGTCTATTTCAGCCTAGAAGAAAAGATAATGAGTAAAACAAATTGCGTTGT
TTAAAGGATTATAGTGCTGCATTGTCTGAAGTTAGCACCTCTTGGACTGAATCGTTTGTCTAGACTACAT
GTATTACAAAGTCTCTTTGGCAAGATTGCAGCAAGATCATGTGCATATCATCCCATTGTAAAGCGACTTC
AAAAATATGGGAACACAGTTAGTTATTTTTACACAGTTCTTTTTGTTTTTGTGTGTGTGTGCTGTCGCTT
GTCGACAACAGCTTTTTGTTTTCCTCAATGAGGAGTGTTGCTCATTTGTGAGCCTTCATTAACTCGAAGT
GAAATGGTTAAAAATATTTATCCTGTTAGAATAGGCTGCATCTTTTTAACAACTCATTAAAAAACAAAAC
AACTCTGGCTTTTGAGATGACTTATACTAATTTACATTGTTTACCAAGCTGTAGTGCTTTAAGAACACTA
CTTAAAAAGCAAAATAAACTTGGTTTACATTTA

Primers or probes can be designed so that they hybridize under stringent conditions to mutant nucleotide sequences of XPO1, but not to the respective wild-type nucleotide sequences. Primers or probes can also be prepared that are complementary and specific for the wild-type nucleotide sequence of XPO1, but not to any one of the corresponding mutant nucleotide sequences. Alternatively, primers or probes can be designed so that they selectively hybridize to both wild-type and mutant XPO1 nucleotide sequences. In some embodiments, the mutant nucleotide sequences of XPO1 may be a missense mutation, or an alteration, that results in the elevated expression and/or activity of XPO1 (i.e., gain of function mutations). Exemplary gain of function mutations of XPO1 include, but are not limited to E571, R749, and D624.

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torrent™ (Life Technologies, Carlsbad, CA) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Chemotherapeutic Agents of the Present Technology

Chemotherapeutic agents can be grouped into several general classes based on their mechanism of action: taxanes, alkylating agents, antitumor antibiotics, topoisomerase inhibitors (e.g., topoisomerase I or II inhibitors), endoplasmic reticulum stress inducing agents, antimetabolites, and mitotic inhibitors. While chemotherapeutic agents can be of substantial therapeutic benefit in many patients, their effectiveness is limited in many types of cancer. Moreover, chemotherapy resistance remains a major hindrance in cancer treatment. In order to improve clinical outcomes, a deeper understanding of the mechanisms that regulate chemotherapy sensitivity and resistance is necessary.

The present disclosure demonstrates that various classes of chemotherapeutic agents (having different mechanisms of action) exert synergistic anti-tumor effects when combined with an XPO1 inhibitor of the present technology.

Examples of suitable chemotherapeutic agents useful in the methods of the present technology include, but are not limited to, antimetabolites, DNA alkylating agents, platinum agents, taxanes, topoisomerase inhibitors, endoplasmic reticulum stress inducing agents, anti-tumor antibiotics, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

### XPO1 Inhibitors of the Present Technology

In one aspect, the present disclosure provides XPO1-specific inhibitory nucleic acids comprising a nucleic acid molecule which is complementary to a portion of an XPO1 nucleic acid sequence (e.g., an XPO1 nucleic acid sequence selected from the group consisting of SEQ ID NOs: 1-9 and 16).

The present disclosure also provides an antisense nucleic acid comprising a nucleic acid sequence that is complementary to and specifically hybridizes with a portion of any one of an XPO1 mRNA (e.g., an XPO1 mRNA sequence selected from the group consisting of SEQ ID NOs: 1-9 and 16), thereby reducing or inhibiting expression of one or more nucleic acids encoding exportin-1. The antisense nucleic acid may be antisense RNA, or antisense DNA. Antisense nucleic acids based on the known XPO1 gene sequence can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence. Accordingly, an antisense nucleic acid can form hydrogen bonds with a sense nucleic acid. The antisense nucleic acid can be complementary to an entire XPO1 coding strand, or to a portion thereof, e.g., all or part of the protein coding region (or open reading frame). In some embodiments, the antisense nucleic acid is an oligonucleotide which is complementary to only a portion of the coding region of an XPO1 mRNA. In certain embodiments, an antisense nucleic acid molecule can be complementary to a noncoding region of the XPO1 coding strand. In some embodiments, the noncoding region refers to the 5′ and 3′ untranslated regions that flank the coding region and are not translated into amino acids. For example, the antisense oligonucleotide can be complementary to the region surrounding the translation start site of an XPO1 gene. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-metnylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual 1-units, the strands run parallel to each other (Gaultier et al., Nucleic Acids. Res. 15:6625-6641(1987)). The antisense nucleic acid molecule can also comprise a 2′-O-methylribonucleotide (Inoue et al., Nucleic Acids Res. 15:6131-6148 (1987)) or a chimeric RNA-DNA analogue (Inoue et al., FEBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of an XPO1 mRNA (e.g., an XPO1 mRNA selected from among any one of SEQ ID NOs: 1-9 and 16), thereby reducing or inhibiting expression of one or more nucleic acids encoding exportin-1. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hydridize to a mRNA of a target gene. Following intracellular delivery, the siRNA or shRNA molecule associates with an RNA-induced silencing complex (RISC), which then binds and degrades a complementary target mRNA (such as an XPO1 mRNA).

The present disclosure also provides a ribozyme comprising a nucleic acid sequence that is complementary to and specifically hybridizes with a portion of an XPO1 mRNA (e.g., an XPO1 mRNA selected from among any one of SEQ ID NOs: 1-9 and 16), thereby reducing or inhibiting expression of one or more nucleic acids encoding exportin-1. Ribozymes are catalytic RNA molecules with ribonuclease activity which are capable of cleaving a complementary single-stranded nucleic acid, such as an mRNA. Thus, ribozymes (e.g., hammerhead ribozymes (described in Haselhoff and Gerlach, Nature 334:585-591 (1988))) can be used to catalytically cleave XPO1 transcripts, thereby inhibiting translation of one or more transcripts encoding exportin-1.

A ribozyme having specificity for an XPO1-encoding nucleic acid can be designed based upon an XPO1 nucleic acid sequence disclosed herein. For example, a derivative of a Tetrahymena L-19 IVS RNA can be constructed in which the nucleotide sequence of the active site is complementary to the nucleotide sequence to be cleaved in an XPO1-encoding mRNA. See, e.g., U.S. Pat. Nos. 4,987,071 and 5,116,742. Alternatively, an XPO1 mRNA can be used to select a catalytic RNA having a specific ribonuclease activity from a pool of RNA molecules. See, e.g., Bartel and Szostak (1993) Science 261:1411-1418, incorporated herein by reference.

The present disclosure also provides a synthetic guide RNA (sgRNA) comprising a nucleic acid sequence that is complementary to and specifically hybridizes with a portion of an XPO1 nucleic acid (e.g., an XPO1 nucleic acid selected from among any one of SEQ ID NOs: 1-9 and 16). Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of an XPO1 nucleic acid (e.g., an XPO1 nucleic acid selected from among any one of SEQ ID NOs: 1-9 and 16), and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465

In some embodiments, a synthetic guide RNA is a single RNA represented as comprising the following elements: 5′-X1-X2-Y-Z-3′ where X1 and X2 represent the crRNA segment, where X1 is the targeting sequence that binds to a portion of an XPO1 nucleic acid (e.g., an XPO1 nucleic acid selected from among any one of SEQ ID NOs: 1-9 and 16), X2 is a stem sequence the hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of an XPO1 nucleic acid (e.g., an XPO1 nucleic acid selected from among any one of SEQ ID NOs: 1-9 and 16), X2 is a stem sequence the hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator: (a)

(SEQ ID NO: 10)
NNNNNNNNNNNNNNNNNNNNgtttttgtactctcaagatttaGAAAtaaa
tcttgcagaagctacaaagataaggcttcatgccgaaatcaacaccctgt
cattttatggcagggtgttttcgttatttaaTTTTTT;
(b)
(SEQ ID NO: 11)
NNNNNNNNNNNNNNNNNNNNgtttttgtactctcaGAAAtgcagaagcta
caaagataaggcttcatgccgaaatcaacaccctgtcattttatggcagg
gtgttttcgttatttaaTTTTTT;
(c)
(SEQ ID NO: 12)
NNNNNNNNNNNNNNNNNNNNgtttttgtactctcaGAAAtgcagaagcta
caaagataaggcttcatgccgaaatcaacaccctgtcattttatggcagg
gtgtTTTTTT;
(d)
(SEQ ID NO: 13)
NNNNNNNNNNNNNNNNNNNNgttttagagctaGAAAtagcaagttaaaat
aaggctagtccgttatcaacttgaaaaagtggcaccgagtcggtgcTTTT
TT;
(e)
(SEQ ID NO: 14)
NNNNNNNNNNNNNNNNNNNNgttttagagctaGAAATAGcaagttaaaat
aaggctagtccgttatcaacttgaaaaagtgTTTTTTT;
and
(f)
(SEQ ID NO: 15)
NNNNNNNNNNNNNNNNNNNNgttttagagctagAAATAGcaagttaaaat
aaggctagtccgttatcaTTTTTTTT.

Selection of suitable oligonucleotides for use in as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

Aptamers are macromolecules composed of nucleic acid (e.g., RNA, DNA) that bind tightly to a specific molecular target (e.g., exportin-1 polypeptide or an epitope thereof). A particular aptamer may be described by a linear nucleotide sequence and is typically about 15-60 nucleotides in length. In some embodiments, aptamers are modified to dramatically reduce their sensitivity to degradation by enzymes in the blood for use in in vivo applications. In addition, aptamers can be modified to alter their biodistribution or plasma residence time.

Selection of aptamers that can bind a exportin-1 polypeptide or a fragment thereof can be achieved through methods known in the art. For example, aptamers can be selected using the SELEX (Systematic Evolution of Ligands by Exponential Enrichment) method (Tuerk, C., and Gold, L., Science 249:505-510 (1990); Jayasena, S. D. Clin. Chem. 45:1628-1650 (1999)).

In another aspect, the present disclosure provides pharmacological inhibitors of XPO1 including, but not limited to leptomycin B (LMB), PKF050-638, CBS9106, selective inhibitors of nuclear transport (SINE) compounds (e.g., KPT-185, KPT-249, KPT-251, KPT-276, KPT-335, KPT-330/Selinexor, SL-801 (felezonexor) and KPT-8602/Eltanexor).

Anti-exportin-1 neutralizing antibodies may also be employed in the methods disclosed herein. Such antibodies include, but are not limited to, polyclonal antibodies; monoclonal antibodies or antigen binding fragments thereof, modified antibodies such as chimeric antibodies, reshaped antibodies, humanized antibodies, or fragments thereof (e.g., Fv, Fab′, Fab, F(ab′)₂); or biosynthetic antibodies, e.g., single chain antibodies, single domain antibodies (DAB), Fvs, or single chain Fvs (scFv). Methods of making and using polyclonal and monoclonal antibodies are well known in the art, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)2 fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer Verlag (Dec. 15, 2000; 1st edition).

Examples of anti-exportin-1 antibody agents useful in the methods disclosed herein include, but are not limited to monoclonal antibodies, human antibodies, humanized antibodies, multi-specific antibodies, bispecific antibodies, camelised antibodies, chimeric antibodies, antibody fragments (e.g., Fab, F(ab′)2, Fab′, scFv, Fv, Fd, dAB), single domain antibodies (e.g., nanobody, single domain camelid antibody), scFv-Fc, VNAR fragments, bispecific T-cell engager (BITE) antibodies, minibodies, antibody drug conjugates, fusion polypeptides, disulfide-linked Fvs (sdFvs), intrabodies, and anti-idiotypic antibodies. Any anti-exportin-1 antibody agents known in the art are useful in the methods disclosed herein.

Formulations Including the XPO1 Inhibitors and/or the Chemotherapeutic Agents of the Present Technology

The pharmaceutical compositions of the present technology can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain solvents, diluents, and other liquid vehicles, dispersion or suspension aids, surface active agents, pH modifiers, isotonic agents, thickening or emulsifying agents, stabilizers and preservatives, solid binders, lubricants and the like, as suited to the particular dosage form desired. In certain embodiments, the compositions disclosed herein are formulated for administration to a mammal, such as a human.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

In order to prolong the effect of a compound of the present disclosure, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings, release controlling coatings and other coatings well known in the pharmaceutical formulating art. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

The active compounds can also be in micro-encapsulated form with one or more excipients as noted above. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or in a certain part of the intestinal tract, optionally, in a delayed manner. Examples of embedding compositions that can be used include polymeric substances and waxes.

### Theranostic Methods of the Present Technology

In one aspect, the present disclosure provides a method for detecting chemoresistant SCLC tumors in a patient in need thereof comprising detecting the presence of at least one mutation that results in elevated expression or activity of exportin-1 in a biological sample obtained from the patient, and/or detecting mRNA and/or polypeptide expression and/or activity levels of exportin-1 in a biological sample obtained from the patient that are elevated compared to a control sample obtained from a healthy subject or a predetermined threshold. The at least one mutation may be detected using any nucleic acid detection assay known in the art such as next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In certain embodiments, the at least one mutation may be an XPO1 missense mutation (e.g., E571, R749, and D624), or an increased copy number of XPO1 gene. In some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry. Additionally or alternatively, in some embodiments, the biological sample comprises polypeptides, genomic DNA, cDNA, RNA, and/or mRNA.

In one aspect, the present disclosure provides a method for selecting a small cell lung cancer (SCLC) patient that has received or is receiving chemotherapy for treatment with an XPO1 inhibitor comprising (a) detecting the presence of at least one mutation that results in elevated expression or activity of exportin-1 in a biological sample obtained from the SCLC patient; and (b) administering an effective amount of an XPO1 inhibitor to the SCLC patient. In some embodiments, the XPO1 inhibitor is separately, sequentially or simultaneously administered with the chemotherapy. In another aspect, the present disclosure provides a method for sensitizing a SCLC patient to chemotherapy comprising administering to the SCLC patient an effective amount of an XPO1 inhibitor separately, sequentially or simultaneously with the chemotherapy, wherein the SCLC patient comprises at least one mutation that results in elevated expression or activity of exportin-1, optionally wherein the at least one mutation is detected in a biological sample obtained from the SCLC patient. The at least one mutation may be detected using any nucleic acid detection assay known in the art such as next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In some embodiments, the biological sample comprises genomic DNA, cDNA, RNA, and/or mRNA. In certain embodiments, the at least one mutation may be an XPO1 missense mutation (e.g., E571, R749, and D624), or an increased copy number of XPO1 gene.

In yet another aspect, the present disclosure provides a method for sensitizing a SCLC patient to chemotherapy comprising administering to the SCLC patient an effective amount of an XPO1 inhibitor separately, sequentially or simultaneously with the chemotherapy, wherein mRNA and/or polypeptide expression and/or activity levels of exportin-1 in a biological sample obtained from the SCLC patient are elevated compared to that observed in a control sample obtained from a healthy subject or a predetermined threshold. In some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any and all embodiments of the methods disclosed herein, the biological sample obtained from the SCLC patient comprises biopsied tumor tissue, whole blood, plasma, or serum.

In one aspect, the present disclosure provides a method for treating SCLC in a patient in need thereof comprising administering to the patient an effective amount of an XPO1 inhibitor and an effective amount of chemotherapy. The XPO1 inhibitor and the chemotherapy may be administered separately, sequentially, or simultaneously. Additionally or alternatively, in some embodiments, exportin-1 mRNA and/or polypeptide expression and/or activity levels in the patient are elevated compared to a healthy subject or a predetermined threshold.

Examples of XPO1 inhibitors useful in any and all embodiments of the present technology include, but are not limited to leptomycin B (LMB), PKF050-638, CBS9106, a selective inhibitors of nuclear transport (SINE) compound, an inhibitory nucleic acid targeting XPO1, and an anti-exportin-1 neutralizing antibody. Examples of SINE compounds include, but are not limited to KPT-185, KPT-249, KPT-251, KPT-276, KPT-335, KPT-330 (Selinexor), SL-801 (felezonexor), or KPT-8602 (Eltanexor). In some embodiments, the inhibitory nucleic acid targeting XPO1 is a shRNA, a siRNA, a sgRNA, a ribozyme, or an anti-sense oligonucleotide.

In any of the preceding embodiments of the methods disclosed herein, the patient has not previously received chemotherapy, or is suffering from chemoresistant SCLC. Additionally or alternatively, in certain embodiments, the patient has a SCLC subtype selected from among ASCL1^{high}, NEUROD1^{high} POU2F3^{high} and YAP^{high}. Additionally or alternatively, in some embodiments, the patient exhibits stage I, stage II, stage III or stage IV SCLC.

In any and all embodiments of the methods disclosed herein, the chemotherapy may comprise one or more chemotherapeutic agents. Examples of chemotherapeutic agents useful in the methods of the present technology include, but are not limited to antimetabolites, DNA alkylating agents, platinum agents, taxanes, topoisomerase inhibitors, endoplasmic reticulum stress inducing agents, anti-tumor antibiotics, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and combinations thereof.

In any of the preceding embodiments of the methods disclosed herein, the XPO1 inhibitor is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically. Additionally or alternatively, in some embodiments of the methods disclosed herein, the one or more chemotherapeutic agents are administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically. In any and all embodiments of the methods disclosed herein, the patient is human.

In therapeutic applications, compositions or medicaments comprising a XPO1 inhibitor disclosed herein and/or chemotherapeutic agents are administered to a subject suspected of, or already suffering from SCLC in an amount sufficient to cure, or at least partially arrest, the symptoms of the disease, including its complications and intermediate pathological phenotypes in development of the disease.

Subjects diagnosed with SCLC can be identified by any or a combination of diagnostic or prognostic assays known in the art.

In some embodiments, subjects suffering from SCLC that are treated with the XPO1 inhibitor and chemotherapy will show amelioration or elimination of one or more of the following symptoms: cough, coughing up blood, shortness of breath, chest pain worsened by deep breathing, hoarse voice, difficulty swallowing, swelling of the face and hands, headache, blurred vision, nausea, vomiting, weakness of limbs, seizures, back pain, loss of bowel or bladder function, bone pain, pain in the upper right abdomen region, fatigue, loss of appetite, muscle weakness, trouble with balance or walking, etc.

In certain embodiments, subjects suffering from SCLC that are treated with the XPO1 inhibitor and chemotherapy will show increased levels of apoptosis and DNA damage in SCLC tumors and/or reduced XPO1 activity levels compared to untreated subjects suffering from SCLC or subjects receiving monotherapy with either the XPO1 inhibitor or chemotherapy.

In some embodiments, the XPO1 inhibitor or chemotherapy is administered one, two, three, four, or five times per day. In some embodiments, the XPO1 inhibitor or chemotherapy is administered more than five times per day. Additionally or alternatively, in some embodiments, the XPO1 inhibitor or chemotherapy is administered every day, every other day, every third day, every fourth day, every fifth day, or every sixth day. In some embodiments, the XPO1 inhibitor or chemotherapy is administered weekly, bi-weekly, tri-weekly, or monthly. In some embodiments, the XPO1 inhibitor or chemotherapy is administered for a period of one, two, three, four, or five weeks. In some embodiments, the XPO1 inhibitor or chemotherapy is administered for six weeks or more. In some embodiments, the XPO1 inhibitor or chemotherapy is administered for twelve weeks or more. In some embodiments, the XPO1 inhibitor or chemotherapy is administered for a period of less than one year. In some embodiments, the XPO1 inhibitor or chemotherapy is administered for a period of more than one year. In some embodiments, the XPO1 inhibitor or chemotherapy is administered throughout the subject's life.

In some embodiments of the methods of the present technology, the XPO1 inhibitor or chemotherapy is administered daily for 1 week or more. In some embodiments of the methods of the present technology, the XPO1 inhibitor or chemotherapy is administered daily for 2 weeks or more. In some embodiments of the methods of the present technology, the XPO1 inhibitor or chemotherapy is administered daily for 3 weeks or more. In some embodiments of the methods of the present technology, the XPO1 inhibitor or chemotherapy is administered daily for 4 weeks or more. In some embodiments of the methods of the present technology, the XPO1 inhibitor or chemotherapy is administered daily for 6 weeks or more. In some embodiments of the methods of the present technology, the XPO1 inhibitor or chemotherapy is administered daily for 12 weeks or more. In some embodiments, the XPO1 inhibitor or chemotherapy is administered daily throughout the subject's life.

### Modes of Administration and Effective Dosages

Any method known to those in the art for contacting a cell, organ or tissue with an XPO1 inhibitor and/or chemotherapeutic agent may be employed. Suitable methods include in vitro, ex vivo, or in vivo methods. In vivo methods typically include the administration of an XPO1 inhibitor and/or chemotherapeutic agent, such as those described herein, to a mammal, suitably a human. When used in vivo for therapy, the XPO1 inhibitor and/or chemotherapeutic agent are administered to the subject in effective amounts (i.e., amounts that have desired therapeutic effect). The dose and dosage regimen will depend upon the degree of the disease symptoms in the subject, the characteristics of the particular XPO1 inhibitor and/or chemotherapeutic agent, e.g., its therapeutic index, the subject, and the subject's history.

The effective amount may be determined during pre-clinical trials and clinical trials by methods familiar to physicians and clinicians. An effective amount of an XPO1 inhibitor and/or chemotherapeutic agent useful in the methods may be administered to a mammal in need thereof by any of a number of well-known methods for administering pharmaceutical compounds. The XPO1 inhibitor and/or chemotherapeutic agent may be administered systemically or locally.

The XPO1 inhibitor and/or chemotherapeutic agent can be incorporated into pharmaceutical compositions for administration, singly or in combination, to a subject for the treatment or prevention of a disorder described herein. Such compositions typically include the active agent and a pharmaceutically acceptable carrier. As used herein, the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

In some embodiments, the XPO1 inhibitor or chemotherapeutic agent described herein is administered by a parenteral route or a topical route.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The XPO1 inhibitor and/or chemotherapeutic agent described herein can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, isotonic agents are included, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, compositions including the XPO1 inhibitor and/or chemotherapeutic agent of the present technology can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of an XPO1 inhibitor and/or chemotherapeutic agent of the present technology as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

AN XPO1 inhibitor and/or chemotherapeutic agent of the present technology can be formulated in a carrier system. The carrier can be a colloidal system. The colloidal system can be a liposome, a phospholipid bilayer vehicle. In one embodiment, the therapeutic XPO1 inhibitor and/or chemotherapeutic agent is encapsulated in a liposome while maintaining structural integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the XPO1 inhibitor and/or chemotherapeutic agent can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the XPO1 inhibitor and/or chemotherapeutic agent are prepared with carriers that will protect the XPO1 inhibitor and/or chemotherapeutic agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The XPO1 inhibitor and/or chemotherapeutic agent can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi et al., Cancer Lett., 100:63-69 (1996), describes the use of fusogenic liposomes to deliver a protein to cells both in vivo and in vitro.

Dosage, toxicity and therapeutic efficacy of the XPO1 inhibitor and/or chemotherapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. In some embodiments, the XPO1 inhibitor and/or chemotherapeutic agent exhibit high therapeutic indices. While the XPO1 inhibitor and/or chemotherapeutic agent that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies within a range of circulating concentrations that include the ED50 with little or no toxicity. The dosage may vary within this range depending upon the dosage form employed and the route of administration utilized. For any XPO1 inhibitor and/or chemotherapeutic agent, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the XPO1 inhibitor and/or chemotherapeutic agent, sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example, dosages can be 1 mg/kg body weight or 10 mg/kg body weight every day, every two days or every three days or within the range of 1-10 mg/kg every week, every two weeks or every three weeks. In one embodiment, a single dosage of an XPO1 inhibitor and/or chemotherapeutic agent ranges from 0.001-10,000 micrograms per kg body weight. In one embodiment, the XPO1 inhibitor and/or chemotherapeutic agent concentrations is in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, and until the subject shows partial or complete amelioration of symptoms of disease. Thereafter, the patient can be administered a prophylactic regime.

In some embodiments, a therapeutically effective amount of an XPO1 inhibitor and/or chemotherapeutic agent may be defined as a concentration of an XPO1 inhibitor and/or chemotherapeutic agent at the target tissue of 10⁻¹² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue. In some embodiments, the doses are administered by single daily or weekly administration, but may also include continuous administration (e.g., parenteral infusion or transdermal application). In some embodiments, the dosage of the XPO1 inhibitor and/or chemotherapeutic agent of the present technology is provided at a “low,” “mid,” or “high” dose level. In one embodiment, the low dose is provided from about 0.0001 to about 0.5 mg/kg/h, suitably from about 0.001 to about 0.1 mg/kg/h. In one embodiment, the mid-dose is provided from about 0.01 to about 1.0 mg/kg/h, suitably from about 0.01 to about 0.5 mg/kg/h. In one embodiment, the high dose is provided from about 0.5 to about 10 mg/kg/h, suitably from about 0.5 to about 2 mg/kg/h.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

### Kits of the Present Technology

The present disclosure provides kits for treating SCLC (e.g., chemonaive or chemoresistant SCLC) comprising an XPO1 inhibitor disclosed herein, and instructions for treating SCLC (e.g., chemonaive or chemoresistant SCLC). In some embodiments, the kits further comprise one or more chemotherapeutic agents. When simultaneous administration is contemplated, the kit may comprise an XPO1 inhibitor and a chemotherapeutic agent that has been formulated into a single pharmaceutical composition such as a tablet, or as separate pharmaceutical compositions. When the XPO1 inhibitor and the chemotherapeutic agent are not administered simultaneously, the kit may comprise an XPO1 inhibitor and a chemotherapeutic agent that has been formulated as separate pharmaceutical compositions either in a single package, or in separate packages.

Examples of suitable chemotherapeutic agents include, but are not limited to, antimetabolites, alkylating agents, platinum agents, taxanes, topoisomerase inhibitors, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

In any and all embodiments of the kits of the present technology, the XPO1 inhibitor may be selected from the group consisting of leptomycin B (LMB), PKF050-638, CBS9106, a selective inhibitors of nuclear transport (SINE) compound, an inhibitory nucleic acid targeting XPO1, and an anti-exportin-1 neutralizing antibody. Examples of SINE compounds include, but are not limited to KPT-185, KPT-249, KPT-251, KPT-276, KPT-335, KPT-330 (Selinexor), SL-801 (felezonexor), or KPT-8602 (Eltanexor). In some embodiments, the inhibitory nucleic acid targeting XPO1 is a shRNA, a siRNA, a sgRNA, a ribozyme, or an anti-sense oligonucleotide.

The kits may further comprise pharmaceutically acceptable excipients, diluents, or carriers that are compatible with one or more kit components described herein. Optionally, the above described components of the kits of the present technology are packed in suitable containers and labeled for the treatment of SCLC (e.g., chemonaive or chemoresistant SCLC). In some embodiments, the SCLC subtype is ASCL1^{high}, NEUROD1^{high}, YAP^{high} or POU2F3^{high}.

The kits may optionally include instructions customarily included in commercial packages of therapeutic products, that contain information about, for example, the indications, usage, dosage, manufacture, administration, contraindications and/or warnings concerning the use of such therapeutic products.

### EXAMPLES

The present technology is further illustrated by the following Examples, which should not be construed as limiting in any way.

### Example 1: Methods and Materials

### CRISPR Screening

Cells were transduced at low MOI (˜30% transduction efficiency) with an in-house made druggable genome library, and transduced cells were selected with puromycin, as described in Wohlhieter, C. A. et al., Cell Rep. 33: 108444 (2020), which is incorporated by reference in its entirety herein. After antibiotic selection, cells were split in 6 flasks with enough number of cells to maintain a minimum of 1000× library representation. 3 flasks (technical replicates) were left untreated and 3 flasks were treated with cisplatin G120 concentration. The performance of the screen, gDNA extraction, NGS PCR, sequencing and analyses were performed as described in Wohlhieter, C. A. et al., Cell Rep. 33: 108444 (2020).

### Monotherapy Cytotoxicity Assay (G150)

GI50 assays were performed as described in Quintanal-Villalonga, A. et al., EBioMedicine 53: 1-15 (2020), which is incorporated by reference in its entirety herein. Viability was assessed with the CellTiter-Glo 2.0 Assay (Promega, Madison, WI, G9242).

### Plasmid Vectors and Transductions

To generate Cas9-expressing cell lines, these were spin-transduced with lentiviral particles made out of a lentiviral plasmid designed to constitutively express Cas9 (#125592, Addgene, Watertown, MA) as described in Hulton, C. H. et al., Nat. Cancer 1: 359-369 (2020), which is incorporated by reference in its entirety herein, and selected with blasticidin 3 μg/mL.

Cells were spin-transduced as described in Hulton, C. H. et al., Nat. Cancer 1: 359-369 (2020) with lentiviral particles made out of lentiviral LV04 vectors expressing sgRNAs for XPO1 (#HSPD0000044805 and #HSPD0000044808 on, Sigma-Aldrich, St. Louis, MO) or the respective control vector expressing a safe targeting sgRNA with GFP (#HSCONTROL_AAVS1 on LV04, Sigma-Aldrich, St. Louis, MO) or BFP (#HSCONTROL_AAVS1 on LV03, Sigma-Aldrich, St. Louis, MO), or a vector overexpressing XPO1 tagged with GFP (#RC206004L4, Origene, Rockville, MD).

### Clonal Competition Assays

The clonal competition assays in vitro were performed as described in Hulton, C. H. et al., Nat. Cancer 1: 359-369 (2020). Cells were transduced separately with vectors to express an XPO1-targeting sgRNA and BFP or a safe targeting sgRNA and GFP, and then mixed together. These were divided in technical replicates for two conditions: untreated and treated with cisplatin GI20. The percentages of each population (determined by expression of GFP or BFP by flow cytometry, as described in Hulton, C. H. et al., Nat. Cancer 1: 359-369 (2020)) was normalized to day 0 for each time point.

The in vivo clonal competition assay was performed as described in Hulton, C. H. et al., Nat. Cancer 1: 359-369 (2020). The inducible Cas9-expressing PDX model Lx33 was transduced with the vectors above indicated separately and re-engrafted in immunocompromised mice. When at 1000 mm³, tumors transduced with each vector were collected and dissociated, mixed 1:1 and re-engrafted into 5 mice per condition, including (1) untreated, (2) doxycycline-treated, (3) cisplatin-treated (2 mg/kg i.p. once/week) and (4) cisplatin- and doxycycline treated. Doxycycline was provided as chow (625 ppm). When control tumors reached 1000 mm³, all tumors were collected, dissociated and analyzed by flow cytometry. Percentages of the GFP and BFP populations were normalized to those of the control untreated condition.

### Synergy Assays

Cells were seeded in 96-well plates (1500 cells/well) and treated with the concentrations of cisplatin, Selinexor and KPT-185 indicated for 5 days. Then, cell viability was assessed with CellTiter-Glo 2.0 Assay (Promega, Madison, WI, G9242) and normalized to the untreated wells. Synergy was calculated using the HSA method using the SynergyFinder web application (2.0) as described in Ianevski, A., Giri, A. K. & Aittokallio, T., Nucleic Acids Res. 48: W488-W493 (2021).

### Rescue Assay

Cells were spin-transduced with lentiviruses made out of an inducible Cas9-expressing vector described in Hulton, C. H. et al., Nat. Cancer 1: 359-369 (2020) and with lentiviruses to express the XPO1 sgRNA or the safe-targeting sgRNA described above, at high MOI. Cells were treated with doxycycline (0.5 μg/mL) for 10 days to induce Cas9 expression and allow XPO1 targeting, and then, high CD4-expressing cells (high Cas9-expressing cells) were sorted as described in Hulton, C. H. et al., Nat. Cancer 1: 359-369 (2020). Sorted cells were cultured with no doxycycline, to turn off Cas9 expression and allow leftover Cas9 to be degraded. Then, cells were transduced with lentiviruses made out of the GFP or XPO1-GFP expressing vectors described above and transduced cells were selected with neomycin (1 mg/mL). Then, cells were treated with cisplatin (GI20) for 5 days, and viability was measured. Viability of the cisplatin-treated conditions were normalized to their matched untreated condition.

### Propidium Iodide Annexin V Assays

Parental cells were treated with cisplatin (GI20), Selinexor (GI20) or their combination for 3 days. Alternatively, control and XPO1 CRISPR-KO cells were treated with cisplatin (G120) for 3 days. Then, cells were collected and stained with the APC Annexin V Apoptosis Detection Kit with PI (#640932, Biolegend, San Diego, CA) and apoptosis was analyzed by flow cytometry as previously described in Shrestha, C. L. et al., PLoS One 12: 1-17 (2017).

### Immunoblot

Protein extraction and western blot were performed as previously described in Gardner, E. E. et al., Cancer Cell 31: 286-299 (2017), which is incorporated by reference in its entirety herein. Antibodies for pγH2AX (#39117, Active Motif, Carlsbad, CA), pATR (#53217, Cell Signaling Technology, Danvers, MA, Danvers, MA), ATR (#13934, Cell Signaling Technology, Danvers, MA, Danvers, MA), pATM (#31068, Cell Signaling Technology, Danvers, MA, Danvers, MA), ATM (#2873, Cell Signaling Technology, Danvers, MA, Danvers, MA), pChk2 (#12298, Cell Signaling Technology, Danvers, MA, Danvers, MA), Chk2 (#2662, Cell Signaling Technology, Danvers, MA, Danvers, MA), MSH2 (#2017, Cell Signaling Technology, Danvers, MA, Danvers, MA), MLH1 (#3515, Cell Signaling Technology, Danvers, MA, Danvers, MA), XPO1 (#46249, Cell Signaling Technology, Danvers, MA, Danvers, MA), and actin (#3700, Cell Signaling Technology, Danvers, MA, Danvers, MA). Quantifications were performed with the Image Studio software (Version 3.1, LI-COR Biosciences, Lincoln, NE). Immunohistochemistry for Exportin-1 was performed with the Exportin-1 antibody #611833 from BD in TMAs including resected tumor samples form SCLC and NSCLC patients. All study subjects had provided signed informed consent for biospecimen analyses under an Institutional Review Board-approved protocol.

Data from Cancer Cell Line Encyclopedia

XPO1 mRNA expression data from Cancer Cell Line Encyclopedia (CCLE) 15 was downloaded from UCSC Xenabrowser portal (https://xenabrowser.net/) on December 2020.

### In Vivo Treatments

For treatments, 5-10 NOD.Cg-Prkd^{scid} Il2rg^{tm1Wjl}/SzJ (NSG) mice were engrafted per treatment arm and incubated until they reached 100-150 mm³. At that point, mice were randomized into groups and treated with either vehicle, Cisplatin (2 mg/kg i.p. once/week), Etoposide (3 mg/kg i.p. QD×3), Selinexor (10 mg/kg p.o. QD×3), irinotecan (50 mg/kg i.p. WQ), or the combinations of cisplatin+etoposide, cisplatin+Selinexor, cisplatin+etoposide+Selinexor or irinotecan+Selinexor, at the previously mentioned doses. Mice weights and tumor volumes were measured twice a week and mice were sacrificed when tumors reached humane endpoint. All animal experiments were approved by the Memorial Sloan Kettering Cancer Center (MSKCC) Animal Care and Use Committee.

### Example 2: CRISPR Screen Identifies XPO1 as a Target for Cisplatin Sensitization in SCLC

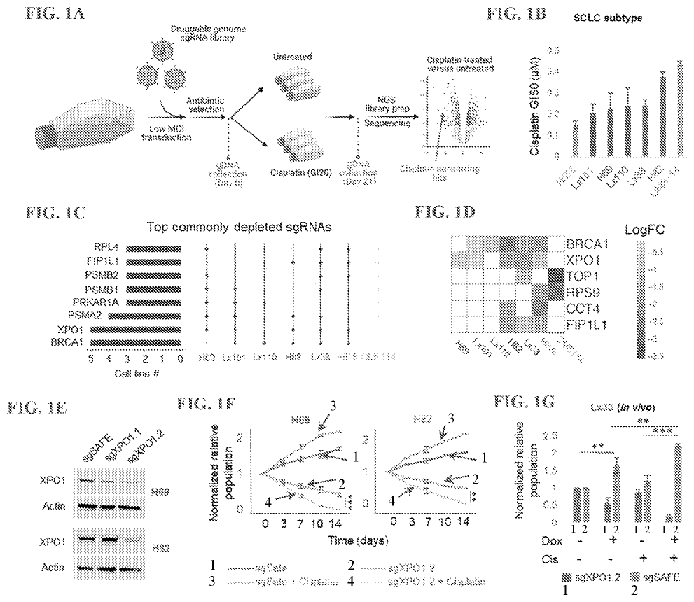

A druggable genome sgRNA library described in Wohlhieter, C. A. et al., Cell Rep. 33, (2020), which is incorporated by herein in its entirety, with ˜3000 sgRNAs targeting ˜750 genes amenable to be targeted therapeutically (with pharmacological inhibitors at late stages of clinical development, or FDA-approved drugs currently in use) was leveraged for the present disclosure. This library was transduced via lentivirus (FIG. 1A) into 4 commercially available SCLC cell lines and 3 cell lines derived from PDXs with limited passages in vitro with constitutive Cas9 expression, with different cisplatin sensitivities and representing the major SCLC subtypes (FIG. 1B). “Drop-out” screens in both a control—untreated- and cisplatin G120-treated conditions were performed for a total of 15 population doublings. Deep sequencing analyses comparing sgRNA abundance of the cisplatin relative to the untreated condition identified hits shared among different cell lines, including BRCA1, depleted in 5 out of 7 cell lines (FIGS. 1C-1D). XPO1, a nuclear export protein mediating export of proteins and mRNAs to the cytoplasm with efficacious targeted inhibitors approved for clinical use in advance diffuse large B cell lymphoma and multiple myeloma, was also found as a hit in 5 out of 7 cell lines (FIGS. 1C-1D), including cell lines from all major SCLC subtypes.

Independent genetic validation using an alternative sgRNA against XPO1 (sgXPO1.2, FIG. 1E) was performed in vitro though clonal competition assays that revealed a significantly accelerated decrease in the XPO1 KO cell population in the cisplatin-treated versus the control untreated condition (FIG. 1F) in two SCLC cell lines belonging to the major SCLC cell lines, and in vivo by the use of an inducible Cas9-expressing SCLC PDX model revealing decreased XPO1 KO cell population in the cisplatin-treated condition at the time of tumor collection (FIG. 1G), suggesting increased cisplatin sensitivity in the XPO1 KO cells.

These results demonstrate that combination therapy with an XPO1 inhibitor and a chemotherapeutic agent sensitizes a SCLC patient to chemotherapy. Accordingly, the combination therapy methods disclosed herein are useful for treating SCLC (e.g., chemonaive or chemoresistant SCLC) in a subject in need thereof.

### Example 3: Synergistic Cytotoxic Effects of XPO1 Inhibition in Combination with Cisplatin

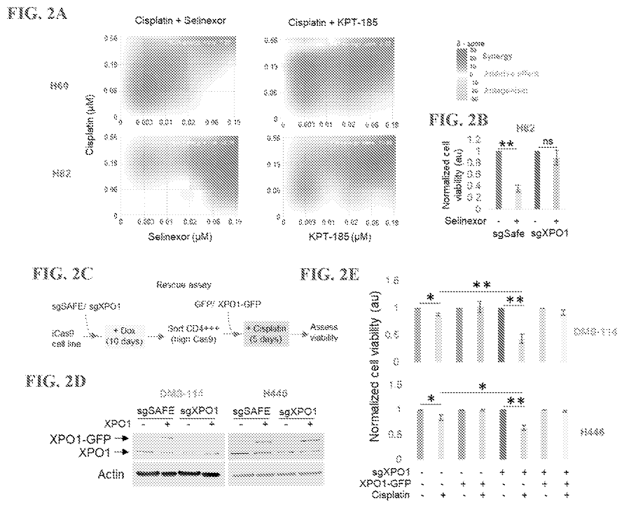

Next, a pharmacological validation of the above findings by assaying combinatorial treatments of cisplatin together with two different XPO1 inhibitors, Selinexor and KPT-185, was performed in two SCLC cell lines (FIG. 2A). Assessment of viability of these models under different combinations of concentrations of cisplatin with either of the XPO1 inhibitors allowed estimation of synergy between the two drugs by HSA synergy score calculation, revealing synergistic effects of the combination of cisplatin with Selinexor (HSA score of 6.55 and 12.29 in H69 and H82, respectively) and KPT-185 (HSA score of 8.52 and 10.68 in H69 and H82, respectively) (FIG. 2A). Selinexor was focused on for further experiments due to its clinical relevance, as its use is approved for clinical use in hematological malignancies.

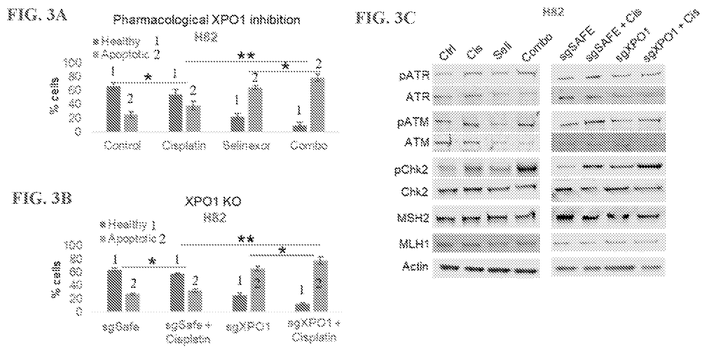

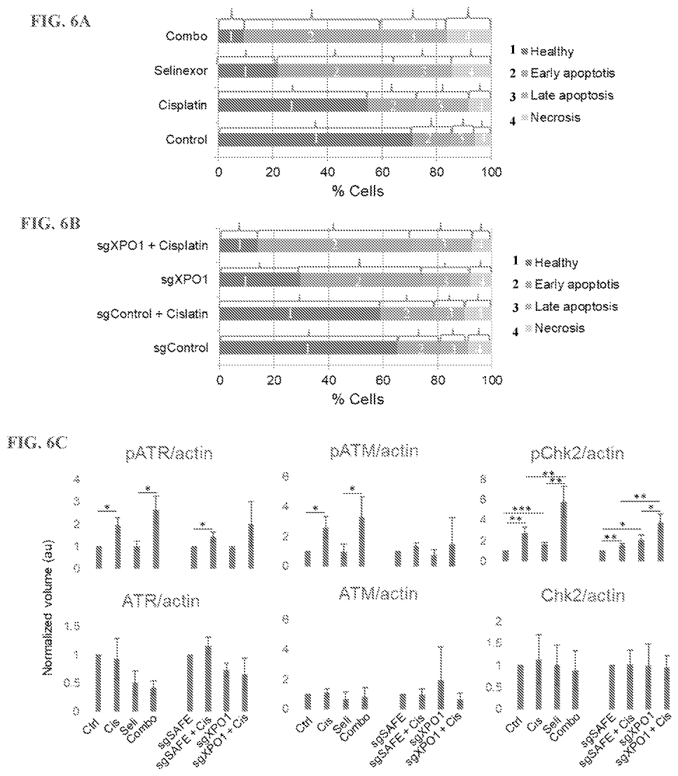

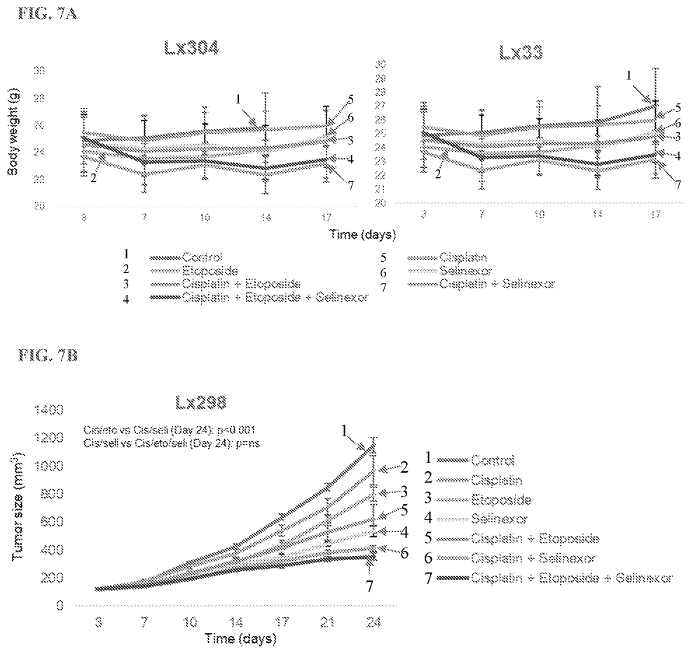

Assessment of cell viability after Selinexor treatment in XPO1-expressing and XPO1-KO isogenic cell lines revealed no effect in viability in the XPO1-KO cell line (FIG. 2B), consistent with high target specificity. Additionally, performance of rescue experiments by re-expression of XPO1 in two XPO1-KO cell lines (FIGS. 2C-2D) revealed that exogenous XPO1 expression rescued the increased cisplatin sensitivity phenotype after XPO1-KO (FIG. 2E), further supporting the role of XPO1 inhibition as cisplatin sensitizer. Propidium iodide/annexin V assays in vitro revealed increased apoptosis after XPO1 pharmacological (FIG. 3A and FIG. 6A) or genetic (FIG. 3B and FIG. 6B) inhibition, further increased by its combination with cisplatin (FIGS. 3A-3B and FIGS. 6A-6B), suggesting that the cytotoxic effects of these combinations are mediated by an induction of early and late apoptosis (FIGS. 3A-3B and FIGS. 6A-6B).

Western blot assessment of the activation of DNA damage response pathways revealed that, even if ATR and ATM were activated after cisplatin treatment, as expected (FIG. 3C and FIG. 6C), combination with XPO1 pharmacological or genetic inhibition did not further increase their activation (FIG. 3C and FIG. 6C). However, the downstream effector Chk2 showed increased activation by cisplatin when XPO1 function was pharmacologically or genetically disrupted (FIG. 3C and FIG. 6C), which consistently with increased pγH2AX levels in the XPO1 deficient conditions (FIG. 3C) was suggestive of increase DNA damage after combination of cisplatin and XPO1 inhibition. Selinexor has been previously involved in sensitization to other DNA damaging agents by downregulating proteins involved in DNA damage repair, including MSH2 and MLH1 (Kashyap, T. et al., Oncotarget 9: 30773-30786 (2018); Ranganathan, P. et al., Clin. Cancer Res. 22: 6142-6152 (2016)). However, reduction in the expression of these two proteins (MSH2 and MLH1) upon Selinexor treatment was not observed (FIG. 3C), suggesting an alternative mechanism of action for Selinexor in the SCLC setting.

This was further confirmed in RNA-seq data of three models treated with cisplatin, Selinexor, or their combination (H69 and H82 in vitro, and Lx33 in vivo), in which selinexor treatment did not show substantial decrease in the expression of these and other DNA damage repair genes assayed, and in which no consistent downregulation of genes involved in chemotherapy-induced DNA damage repair pathways was observed across the models tested. These results demonstrate that (i) exportin-1 inhibition promotes apoptosis in chemotherapy-treated SCLC, and (ii) inhibition of DNA damage repair pathways is not a primary mechanism for exportin-1 inhibition-mediated chemotherapy sensitization in SCLC. Altogether, these results suggest that cell growth inhibition by XPO1 targeting synergizes with cisplatin by increasing DNA damage and apoptosis.

These results demonstrate that combination therapy with an XPO1 inhibitor and a chemotherapeutic agent sensitizes a SCLC patient to chemotherapy. Accordingly, the combination therapy methods disclosed herein are useful for treating SCLC (e.g., chemonaive or chemoresistant SCLC) in a subject in need thereof.

### Example 4: SCLC Tumors Express High Levels of Exportin-1

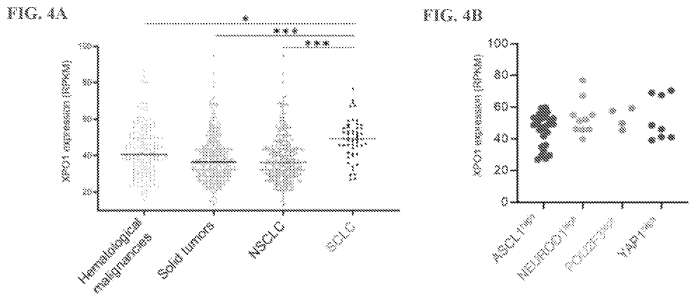

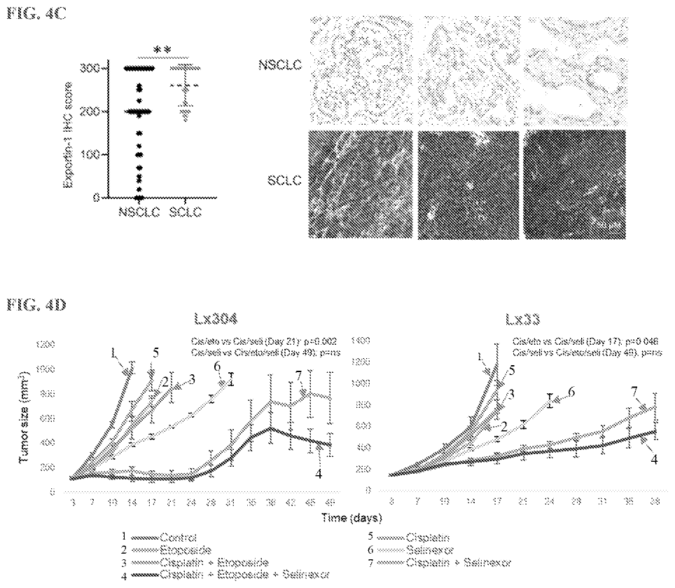

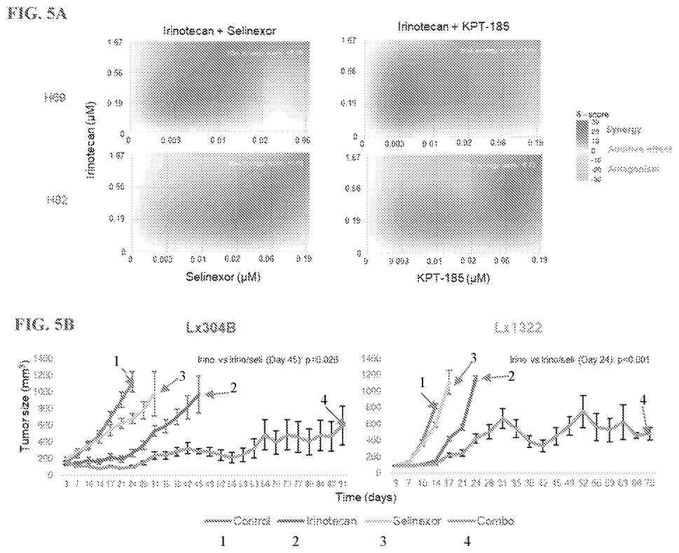

Next, XPO1 expression in SCLC as a potential surrogate for XPO1 inhibitor efficacy was assessed in this setting. Leveraging the CCLE publicly available database (Barretina, J. et al., Nature 483: 603-607 (2012)), high XPO1 mRNA expression was observed in SCLC cell lines (FIG. 4A), superior to cell lines derived from other lung cancer subtypes and from other cancer types, and comparable to those derived from hematological malignancies, the setting where XPO1 inhibitors have shown high efficacy and are currently in clinical use (Azizian, N. G et al., J. Hematol. Oncol. 13: 1-9 (2020); Ranganathan, P. et al., Clin. Cancer Res. 22: 6142-6152 (2016)). Additionally, no significant difference in XPO1 mRNA expression dividing by SCLC subtypes (FIG. 4B) in these cell lines was observed. Assessment of XPO1 protein levels by immunohistochemistry in NSCLC (N=58) and SCLC (N=32) clinical specimens revealed high expression of this protein in all SCLC tumors assessed (FIG. 4C). These results indicate that SCLC tumors exhibit high XPO1 expression levels, independently to the SCLC subtype, superior to other lung cancer subtypes and tumors from other origins, suggesting that XPO1 inhibition may be a potential therapeutic target in SCLC tumors.

These results demonstrate that combination therapy with an XPO1 inhibitor and a chemotherapeutic agent sensitizes a SCLC patient to chemotherapy. Accordingly, the combination therapy methods disclosed herein are useful for treating SCLC (e.g., chemonaive or chemoresistant SCLC) in a subject in need thereof.

### Example 5 Combination of Cisplatin and Selinexor is Highly Effective in Chemonaive SCLC PDXs

The efficacy of the combination of the XPO1 inhibitor Selinexor and cisplatin in two chemonaive SCLC PDXs representing the major SCLC subtypes (Lx304, ASCL1^{high} and Lx33, NEUROD1^{high}) was assessed, and it was compared to the combination of cisplatin and etoposide, the backbone for the current standard of care for first line in this setting (FIG. 4D). In the Lx304 ASCL1^{high} model, neither cisplatin, etoposide or their combination showed high efficacy at reducing tumor growth in this model (FIG. 4D left), and Selinexor slowed down tumor growth to a higher extent than the previously mentioned therapies at the doses used (T/C percentage values at control group endpoint (day 14) of 67.04, 54.59, 51.99 and 38.52% for cisplatin, etoposide, their combination, and Selinexor, respectively). The combination of Selinexor and cisplatin and the triple combination of Selinexor, cisplatin and etoposide showed exquisite efficacy in this model, with 83.42 (p=0.002) and 90.50% (p=0.001) tumor growth reduction compared to the combination of cisplatin and etoposide at day 21 (FIG. 4D left). The triple combination therapy failed to reach statistical significance in terms of tumor growth reduction when compared to the combination of cisplatin and Selinexor (FIG. 4D left). Similar results were observed for the Lx33 NEUROD1^{high} model (FIG. 4D right), with little efficacy of cisplatin, etoposide or their combination, and higher efficacy of Selinexor than the latter three (T/C percentage values at control arm endpoint (day 17) of 79.73, 63.14, 64.39 and 40.99% for cisplatin, etoposide, their combination, and Selinexor, respectively). In this model again the Selinexor double and triple combinations showed high efficacy, with growth reduction of 57.12 (p=0.048) and 78.28% (p=0.011) as compared to the combination of cisplatin and etoposide at day 17) (FIG. 4D right), but similarly to the previous model, the triple combination did not show a significantly higher reduction in tumor growth than the Selinexor and cisplatin combination.

These results demonstrate that combination therapy with an XPO1 inhibitor and a chemotherapeutic agent sensitizes a SCLC patient to chemotherapy. Accordingly, the combination therapy methods disclosed herein are useful for treating SCLC (e.g., chemonaive or chemoresistant SCLC) in a subject in need thereof.

### Example 6: Combination of Irinotecan and Selinexor Shows High Efficacy in Chemorelapsed SCLC PDXs

Next, if XPO1 inhibition may sensitize SCLC to irinotecan, a topoisomerase I inhibitor used in chemorelapsed SCLC as a second line therapy, was determined. Combination of irinotecan with either Selinexor or KPT-185 in vitro was synergistic in terms of growth inhibition in two SCLC cell lines (HSA score of 15.10 and 11.50 for irinotecan and Selinexor, and of 13.60 and 9.73 for irinotecan and KPT-185, in H69 and H82, respectively) (FIG. 5A), and exhibiting higher synergy than the combination of these inhibitors with cisplatin (FIG. 2A).

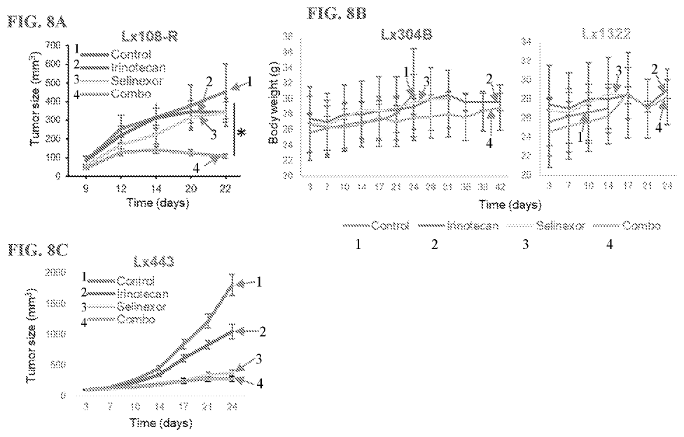

In vivo efficacy assessment of Selinexor with irinotecan in two SCLC PDXs established from chemorelapsed tumors from different subtypes (Lx304B, ASCL1^{high} and Lx1322, non-NE) demonstrated exquisite efficacy of this combination (FIG. 5B), with T/C percentage values of 23.82 and 9.23% at control arm endpoint (day 24) for irinotecan and the combination, respectively, in Lx304B, and of 18.09 and 13.67% (day 14), in Lx1322. The combination was substantially more efficacious than irinotecan monotherapy, significantly delaying tumor growth (70.38 and 63.62% tumor growth reduction at irinotecan arm endpoint for Lx304B (day 45, p=0.026) and Lx1322 (day 24, p<0.001), respectively (FIG. 5B). Additionally, the combination of irinotecan and Selinexor in a cisplatin/etoposide-resistant SCLC PDX generated in vivo (Lx108-R, ASCL1^{high}), right after chemotherapy relapse in vivo (FIG. 8A) was tested, which again showed high efficacy with a T/C percentage of 75.27 and 23.83% at experiment endpoint for irinotecan and the combination, respectively, and growth inhibition of 68.57% of the combination as compared to irinotecan monotherapy (p=0.011).

These results demonstrate that combination therapy with an XPO1 inhibitor and a chemotherapeutic agent sensitizes a SCLC patient to chemotherapy. Accordingly, the combination therapy methods disclosed herein are useful for treating SCLC (e.g., chemonaive or chemoresistant SCLC) in a subject in need thereof.

### Example 7: Suppression of AKT/mTOR Activation May Contribute to Sensitization to Chemotherapy by Exportin-1 Inhibition

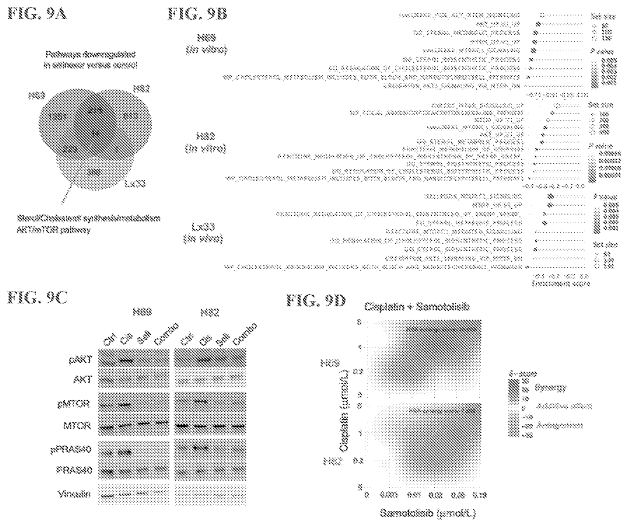

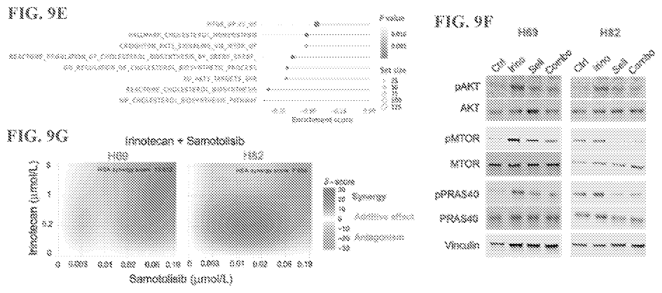

In a broader exploration of pathways affected by exportin-1 inhibition in SCLC, RNA-seq data in two cell line models (in vitro, H69 and H82) and in one PDX model (Lx33, in vivo) with or without selinexor (FIG. 9A) was analyzed. Pathway enrichment analyses was performed on the differentially expressed genes of the selinexor-treated versus control untreated conditions and looked for common dysregulated pathways among these models. No common upregulated pathways were identified, but 14 common pathways downregulated by selinexor monotherapy were observed (FIG. 9A), most of which were related to cholesterol synthesis and the AKT/mTOR signaling pathway.

Downregulation of genes involved in cholesterol synthesis and AKT/mTOR signaling pathways in the cisplatin+selinexor-treated models was observed relative to their respective cisplatin-treated conditions in vitro and in vivo (FIG. 9B). Synergy studies on the combination of cisplatin with lovastatin, a cholesterol synthesis inhibitor, revealed no substantial synergy in this combination, suggesting that cholesterol synthesis downregulation may not be a major player in the sensitization to chemotherapy.

To test whether the AKT/mTOR pathway suppression might in part explain the effects caused by exportin-1 inhibition, the activation of the AKT/mTOR pathway in the in vitro SCLC models was assessed by Western blot (FIG. 9C). Increased phosphorylation of mTOR, AKT, and the AKT/mTOR downstream effector PRAS40 was observed after cisplatin treatment in vitro (FIG. 9C), and consistently, increased AKT activation in vivo. This induction was suppressed by selinexor, consistent with the RNA-seq data (FIG. 9B). Overexpression of a constitutively activated isoform of AKT (myristoylated AKT) reduced sensitivity to cisplatin in vitro, and the combination of the AKT/mTOR inhibitor samotolisib and cisplatin demonstrated synergy in vitro (FIG. 9D) and increased apoptosis, analogously to selinexor and chemotherapy combination. In addition, exogenous AKT/mTOR activation reduced sensitivity to the combination of cisplatin and Selinexor, further supporting the involvement of this signaling axis in the sensitizing phenotype observed.

Similar results were obtained for irinotecan: AKT/mTOR pathway genes were downregulated in the RNA-seq data for selinexor+irinotecan versus irinotecan alone in the PDX model Lx1322 in vivo (FIG. 9E); AKT/mTOR pathway was induced by irinotecan treatment in vitro and in vivo but downregulated when combined with selinexor (FIG. 9F); and the combination of irinotecan with samotolisib (FIG. 9G), but not with lovastatin exerted synergistic effects. Taken together these results suggest that cisplatin and irinotecan induce AKT/mTOR signaling in SCLC and that the sensitization to these agents by exportin-1 inhibition may be mediated in part by its ability to abrogate the induction of this survival pathway.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for selecting a small cell lung cancer (SCLC) patient that has received or is receiving chemotherapy for treatment with an XPO1 inhibitor comprising
(a) detecting the presence of at least one mutation that results in elevated expression or activity of exportin-1 in a biological sample obtained from the SCLC patient; and
(b) administering an effective amount of an XPO1 inhibitor to the SCLC patient,
wherein the chemotherapy comprises a topoisomerase inhibitor, optionally wherein
the XPO1 inhibitor is separately, sequentially or simultaneously administered with the chemotherapy; or
the biological sample obtained from the SCLC patient comprises biopsied tumor tissue, whole blood, plasma, or serum.

2. (canceled)

3. A method for sensitizing a SCLC patient to chemotherapy comprising
administering to the SCLC patient an effective amount of an XPO1 inhibitor separately, sequentially or simultaneously with the chemotherapy, wherein the chemotherapy comprises a topoisomerase inhibitor, and
wherein the SCLC patient comprises at least one mutation that results in elevated expression or activity of exportin-1, optionally wherein the at least one mutation is detected in a biological sample obtained from the SCLC patient; or
wherein mRNA and/or polypeptide expression and/or activity levels of exportin-1 in a biological sample obtained from the SCLC patient are elevated compared to that observed in a control sample obtained from a healthy subject or a predetermined threshold,
optionally wherein the biological sample obtained from the SCLC patient comprises biopsied tumor tissue, whole blood, plasma, or serum.

4. The method of claim 3, wherein the at least one mutation is an XPO1 missense mutation, or an increased copy number of XPO1, optionally wherein the at least one mutation is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

5. (canceled)

6. (canceled)

7. The method of claim 3_, wherein mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH) or wherein polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

8. (canceled)

9. (canceled)

10. (canceled)

11. (canceled)

12. (canceled)

13. The method of claim 1, wherein the XPO1 inhibitor is selected from the group consisting of leptomycin B (LMB), PKF050-638, CBS9106, a selective inhibitors of nuclear transport (SINE) compound, an inhibitory nucleic acid targeting XPO1, and an anti-exportin-1 neutralizing antibody.

14. The method of claim 13, wherein the SINE compound is KPT-185, KPT-249, KPT-251, KPT-276, KPT-335, KPT-330 (Selinexor), SL-801 (felezonexor), or KPT-8602 (Eltanexor).

15. The method of claim 13, wherein the inhibitory nucleic acid targeting XPO1 is a shRNA, a siRNA, a sgRNA, a ribozyme, or an anti-sense oligonucleotide.

16. The method of claim 1, wherein the patient has not previously received chemotherapy.

17. The method of claim 1, wherein the patient is suffering from chemoresistant SCLC.

18. The method of claim 1, wherein the chemotherapy comprises one or more chemotherapeutic agents selected from the group consisting of antimetabolites, DNA alkylating agents, platinum agents, taxanes, tepeisemerase endoplasmic reticulum stress inducing agents, anti-tumor antibiotics, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, ixabepilone, temozolmide, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, cladribine, midostaurin, bevacizumab, oxaliplatin, melphalan, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, ARC, NPC, rubifen, BN80927, DX-8951f, MAG-CPT, amsacnne, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, lamellarin D, amsacrine, ellipticines, aurintricarboxylic acid, HU-331, and combinations thereof.

19. The method of claim 1, wherein the patient has a SCLC subtype selected from among ASCL1^{high}, NEUROD1^{high} POU2F3^{high} and YAP^{high}.

20. The method of claim 1, wherein the patient exhibits stage I, stage II, stage III or stage IV SCLC.

21. The method of claim 1, wherein the XPO1 inhibitor is administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.

22. The method of claim 18, wherein the one or more chemotherapeutic agents are administered orally, intranasally, parenterally, intravenously, intramuscularly, intraperitoneally, intramuscularly, intraarterially, subcutaneously, intrathecally, intracapsularly, intraorbitally, intratumorally, intradermally, transtracheally, intracerebroventricularly, or topically.

23. The method of claim 1, wherein the patient is human.

24. A kit comprising an XPO1 inhibitor, one or more chemotherapeutic agents, and instructions for treating SCLC.

25. (canceled)

26. The method of claim 1, wherein the topoisomerase inhibitor is selected from anthracyclines (e.g., daunorubicin and doxorubicin), irinotecan, etoposide, etoposide phosphate, topotecan, campothecin, 9-nitrocamptothecin, 9-aminocamptothecin, teniposide, epirubicin, idarubicin, SN-38, exatecan, gimatecan, and diflomotecan,
